(19) United States
(12) Patent Application Publication
(10) Pub. No.: US 2024/0366930 A1
(43) Pub. Date: Nov. 7, 2024

(54) WEARABLE BIOELECTRONICS FOR PROGRAMMABLE DELIVERY OF THERAPY

(71) Applicant: The Regents of the University of California, Oakland, CA (US)

(72) Inventors: Marco Rolandi, Santa Cruz, CA (US); Marcella Gomez, Santa Cruz, CA (US); Mircea Teodorescu, Santa Cruz, CA (US); Min Zhao, Sacramento, CA (US); Roslyn Rivkah Isseroff, Sacramento, CA (US)

(73) Assignee: The Regents of the University of California, Oakland, CA (US)

(21) Appl. No.: 18/639,864

(22) Filed: Apr. 18, 2024

Related U.S. Application Data
(60) Provisional application No. 63/514,931, filed on Jul. 21, 2023.
(60) Provisional application No. 63/496,762, filed on Apr. 18, 2023.

(51) Int. Cl. A61N 1/04; A61N 1/36

(52) U.S. Cl. CPC A61N 1/0444; A61N 1/0432; A61N 1/0448; A61N 1/0468; A61N 1/36031

(57) ABSTRACT

A system including a data-driven controller configured to output signals controlling a dose of therapy to a treatment site and in response to feedback comprising data representing a healing state of the treatment site measured by a sensor; and a pumping system coupled to the controller, the pumping system pumping the therapy to the treatment site.

### CROSS REFERENCE TO RELATED APPLICATIONS

This application claims the benefit under 35 U.S.C. Section 119(e) of:

U.S. Provisional Application No. 63/496,762, filed Apr. 18, 2023, by Marco Rolandi, entitled “AUTOMATED AND SIMULTANEOUS ON-CHIP DELIVERY OF CATIONS IN CLOSED LOOP,” attorney's docket 284.0021USP1, and

‘U.S. Provisional Application No. 63/514,931, filed Jul. 21, 2023, by Marco Rolandi and Houpu Li, entitled “WEARABLE BIOELECTRONICS FOR PROGRAMMABLE DELIVERY OF FLUOXETINE THAT ACCELERATES WOUND HEALING,” attorney's docket 284.0021USP2, both of which applications are incorporated by reference herein.

The present invention is related to International PCT application No. PCT/US23/16245 filed Mar. 24, 2023, entitled “BIOELECTRONIC SMART BANDAGE FOR CONTROLLING WOUND PH THROUGH PROTON DELIVERY” which application claims the benefit under 35 U.S.C. Section 119(e) of the following co-pending and commonly-assigned U.S. Provisional Application Ser. No. 63/323,321, filed on Mar. 24, 2022, by Narges Asefifeyzabadi and Marco Rolandi, entitled “BIOELECTRONIC SMART BANDAGE FOR CONTROLLING WOUND PH THROUGH PROTON DELIVERY,” Attorney's Docket Number 284.0011USP1, both of which application is incorporated by reference herein.

### STATEMENT REGARDING FEDERALLY SPONSORED RESEARCH AND DEVELOPMENT

This invention was made with Government support under Grant No. D20AC00003, HR0011364541, awarded by the Defense Advanced Research Projects Agency(DARPA). The Government has certain rights in the invention.

### BACKGROUND OF THE INVENTION

### 1. Field of the Invention

The present invention relates to a system for delivery of a treatment and methods of making the same.

### 2. Description of the Related Art

Bioelectronic devices aim to bridge the gap between biological system and electronics. ^{1,2} Signal transmission in biological system mainly relies on ion fluxes and the movement of biomolecules, such as neurotransmitters, while conventional electronic devices control the movement of electrons and holes. ³⁻⁵ Many tools have been developed to control or monitor the activities of ions and biomolecules with electronic or optical signals for the purpose of diagnostics and therapeutics. ⁶⁻⁸ Iontronic devices generate, store, and transmit signals via modulating the flow and concentration of ions and small molecules. ^{9,10} In analogy to electronics, examples of iontronic components include organic electrochemical transistors (OECTs), ¹¹ organic electronic ion pumps (OEIPs), ¹² ionic diodes, ¹³ transistors, ¹⁴ memristors, ¹⁵ and bipolar membrane junctions. ¹⁶ By manipulating ions and biomolecules directly, iontronic devices extend the domain from electrically excitable cells to all cell types, such as stem cells to guide differentiation ^{17,18} and skin cells for improved wound healing. ^{19,20} Furthermore, iontronic devices can target specific biological processes by using an ion or biomolecule of choice. These include H⁺ for enzyme activities, gene expression, and neuronal function, ²¹Cl⁻ for cancer and birth defects, ²² and gamma-aminobutyric acid (GABA) for suppression of epilepsy. ²

Among these iontronic devices, OEIPs are used to deliver charged ions and biomolecules with high spatiotemporal resolution and dosage precision. ¹² In recent years, OEIPs have been reported to deliver H⁺, K⁺, Ca⁺⁺, and GABA for triggering cell polarization status in vitro, ¹² controlling epileptiform activity in brain slice models, ²³ affecting sensory function in vivo, ²⁴ suppressing pain sensation in awake animals, ²⁵ and modulating plant physiology. ²⁶ Some physiological processes need more than one species cooperation at the same time, such as the fact that the early embryonic face is patterned by H⁺ and K⁺ion gradients. ^{27,28} Jonsson et al. reported a novel ion pump design that controls multiple delivery points with high temporal resolution. ²⁹ What is needed, however, are improved wearable ion pumping systems that can deliver therapy to a treatment site more accurately (e.g., with a spatially and temporally controllable dose). The present invention satisfies this need.

### SUMMARY OF THE INVENTION

The present disclosure describes a system for delivering therapy to a treatment site, comprising: means for delivering a dose of a therapy to a treatment site; a sensor configured for sensing the treatment site and outputting data in response thereto; and a data-driven controller or data-driven computer configured to control the dose in a closed loop by determining a healing state of the treatment site from the data, and using the healing state as feedback to update or determine the dose delivered to the treatment site so that the therapy increases a rate of healing of the treatment site and/or the healing state converges to a desired healing state.

In one embodiment, the device is implemented as a multiple-ion pump that can selectively deliver multiple ionic species simultaneously, including both anions and cations. In one particular implementation, the ion pump is composed of four independent reservoir electrolytes connected to one target electrolyte, in which 36 microelectrodes are independently controlled with 50 μm resolution. We also demonstrate closed-loop control of ionic concentration using a machine-learning algorithm. ¹² Thus, this multi-ion pump not only enables controlling complicated physiological processes that need more than one ionic species but also provides a highly efficient and customized toolbox for fundamental biological research with an intelligent manner.

In another embodiment, the device is implemented as a programmable bioelectronic bandage capable of delivering the drug fluoxetine (brand name: Prozac) as a personalized treatment regimen to accelerate wound healing.

### BRIEF DESCRIPTION OF THE DRAWINGS

Referring now to the drawings in which like reference numbers represent corresponding parts throughout:

FIGS. 1a-1c are illustrations of a multi-ion pump wherein FIG. 1a is a top view of the multiple-ion pump, showing four reference electrodes in the reservoirs (R1, R2, R3, and R4), microelectrodes, and auxiliary electrode in the target, FIG. 1b shows mapping of the 36 microelectrodes in the target: every four microelectrodes form an array to deliver different ionic species. Every four microelectrodes form a group that can control four different ions, and such groups form a 3×3 matrix in the target solution, and FIG. 1c shows the side view of the multi-ion pump with one reservoir as the representative, showing the multilayer structure.

FIG. 2a show fluorescence images of the microelectrodes in the target electrolyte, where the square highlights a group of microelectrodes that are connected to different reservoirs.

FIG. 2b shows [H⁺]change following the applied voltage: when V₁=1.6 V, H⁺ was delivered to the target, inducing [H⁺]going up, and vice versa.

FIG. 2c shows the [Na⁺]change, following the applied voltage: when V₂=1.6 V, Na⁺ was delivered to the target, inducing [Na⁺]going up, and vice versa.

FIG. 2d shows the ⁻[Cl⁻ ]change, following the applied voltage: when V₄=−1.8 V, Cl⁻ was delivered to the target, inducing [Cl⁻ ]going up, and vice versa.

FIG. 2e. Yellow square highlighted one microelectrode where we applied the voltage, and the four rectangles showed the areas whose fluorescence intensity was monitored.

FIG. 2f. The [H⁺]change in the four rectangles highlighted in (e) following the protocol of (b).

FIG. 2g. The [Na⁺]change in the four rectangles highlighted in (e) following the protocol of (c).

FIG. 2h The [Cl⁻ ]change in the four rectangles highlighted in (e) following the same protocol of (d).

FIGS. 3a-3d show control of the multi-ion pump with a machine-learning controller. FIG. 3a is a schematic of the experimental setup; the microelectrode induces an ion concentration change in solution upon the applied voltage Vₓ. Fluorescence signals are captured and fed into a neural network machine learning (ML) algorithm, which attempts to control Vₓ to match a prescribed fluorescence pattern. FIGS. 3b-3d shows real-time control of H⁺, Na⁺, and Cl⁻, respectively, by machine learning with desired targets.

FIGS. 4a-4h. The design and characterization of the wearable bioelectronic bandages. FIG. 4a: The ion pump's working principle is illustrated, where the reservoirs are filled with 0.01M fluoxetine hydrochloride (FlxCl) solution. Under positive V_{Flx}, the Flx⁺cations migrate from the reservoir to the wound under electrophoretic force and exchange with physiological cations. An ion selective AMPSA:PEGDA polyanion hydrogel (light yellow) is negatively charged, acting as a filter that blocks anions such as Cl⁻, while allowing cations such as Flx⁺to pass. FIG. 4b: The electrochemical reaction for the ion pump electrodes is depicted happening at the opposite direction simultaneously. The Ag on the working electrode (gray) becomes oxidized under the electrochemical reaction, absorbs a Cl⁻ from the Flx·HCl solution, releases an electron, and becomes AgCl. This reaction releases an electron to the external circuit and leaves a free Flx⁺ cation. The Flx⁺ then migrates to the wound under electrophoretic force. The cathode reaction consumes an electron and releases a free Cl⁻, which can balance with the incoming cation. FIG. 4c: A CAD model of the ion pump, made from PDMS. The ion pump has four reservoirs for fluoxetine solution, where electrode wires made from Ag and AgCl are inserted and connected to the external circuit through contact pins made of steel. The scale bar is 5 mm. FIG. 4d: CAD models of the wearable bioelectronic bandage device assembly, with the controller module on top and the ion pump on the bottom, are displayed. The scale bar is 5 mm. FIG. 4e: A camera photo of the wearable bioelectronic bandage is presented, and the scale bar is 5 mm. FIG. 4f: The circuit diagram of the controller module is provided. It includes a microcontroller with a built-in clock and analog-to-digital converter (ADC), a memory chip, a digital-to-analog converter (DAC), and resistors for sensing current. FIG. 4g: A camera photo of the ex-vivo test setup for the wearable bioelectronic bandage while the device is in contact with the saline solution inside a PDMS well is shown, and the scale bar is 5 mm. FIG. 4h: The plot of the device's current response to a 2 V (peak to peak) square wave applied with a potentiostat is presented, indicating high temporal resolution.

FIG. 5a depicts the wearable bioelectronic bandage applied to a mouse model.

FIG. 5b is a CAD design of the wearable bioelectronic bandage, consisting of a battery-powered PCB controller module (top) and an ion pump made from PDMS (bottom).

FIG. 5c shows the transit and accumulated dose curve of the programmed delivery of fluoxetine is plotted. The wearable bioelectronic bandage delivers 30 μg of fluoxetine over 6 hours and repeats daily.

FIG. 5d are schematics of the ion pumping process show that the electric field drives the fluoxetine cations (green spheres) to the wound through the ion-selective hydrogel (light yellow).

FIGS. 6a-6c shows in vivo experiment of wearable bioelectronic bandage on mice, wherein FIG. 6a is a photo of a mouse wound model during surgery. The left panel shows the mouse wound sutured with a silicone splint ring, and the right panel shows the wearable bioelectronic bandage fixed on the wound by Tegaderm. Scalebar=5 mm, FIG. 6b shows a mouse maintains normal daily activity while wearing the bioelectronic bandage on its wound, and FIG. 6c is a plot of fluoxetine actuation current over time.

FIG. 6d illustrates healing progression.

FIG. 6e illustrates wound size.

FIG. 6f illustrates wound size as a function of time.

FIGS. 6g-h illustrate wound staining with hematoxylin and eosin to assess wound re-epithelialization.

FIG. 6i treatment of wounds with Flx⁺ delivered from the bioelectronic bandage resulted in a 39.9% (P<0.05) increase in re-epithelialization compared to a control.

FIG. 6j illustrates across 15 mice, the bioelectronic bandage consistently delivered the desired dose with some variability (FIG. 6j, left). This variability allowed qualitative correlation of the dose of Flx⁺ and wound healing as measured by the percentage of re-epithelization (FIG. 6j, right).

FIGS. 7a-7d shows macrophage analysis of the wound, wherein FIG. 7a shows staining of different macrophage subtypes in the wound section. All cells (stained by DAPI, shown in blue), M1 (stained by iNOS, shown in green), M2 (stained by CD206, shown in red), and overall macrophages (stained by F4/80, shown in gray) were imaged and presented, FIG. 7b is an overlay of M1 and M2 staining, showing the distribution and ratio of macrophage subtypes, FIG. 7c is a box plot showing statistics of the M1/M2 ratio, which was 1.80+/−0.62 in the control group and 1.31+/−0.46 for the fluoxetine-treated group. The fluoxetine-treated wounds demonstrated an average of 27.2% decrease in M1/M2 ratio compared to control wounds, n=13 mice, p<0.05, FIG. 7d is a plot of the M1/M2 ratio over time. The blue dot represents the M1/M2 ratio from the control group, and the red dot represents the fluoxetine-treated group. The dashed lines indicate the projected wound age, while the solid line represents the trend plotted from the data in the reference. [23].

FIG. 8. Schematic of an Intelligent wound care management system.

FIG. 9. Schematic of a hardware environment for implementing the methods and operating the devices and systems described herein.

FIG. 10. Schematic of a network environment.

FIG. 11. Flowchart illustrating a method of fabricating a device.

FIG. 12. Flowchart illustrating a method of performing closed loop control with data-driven or data-informed algorithm.

FIG. 13. Flowchart illustrating a method of imaging a wound using computer vision.

### DETAILED DESCRIPTION OF THE INVENTION

In the following description of the preferred embodiment, reference is made to the accompanying drawings which form a part hereof, and in which is shown by way of illustration a specific embodiment in which the invention may be practiced. It is to be understood that other embodiments may be utilized and structural changes may be made without departing from the scope of the present invention.

### Technical Description

The present disclosure describes a system for delivering therapy to a treatment site, comprising: means for delivering a dose of a therapy to a treatment site; a sensor configured for sensing the treatment site and outputting data in response thereto; and a data-driven controller or data-driven computer configured to control the dose in a closed loop by determining a healing state of the treatment site from the data, and using the healing state as feedback to update or determine the dose delivered to the treatment site so that the therapy increases a rate of healing of the treatment site and/or the healing state converges to a desired healing state.

The system can be implemented in many ways, illustrative embodiments of which are described herein.

### First Embodiment: Multi-Ion Electrophoretic Pump for Simultaneous On-Chip Delivery of H⁺, Na⁺, and Cl⁻

a. Device Operation

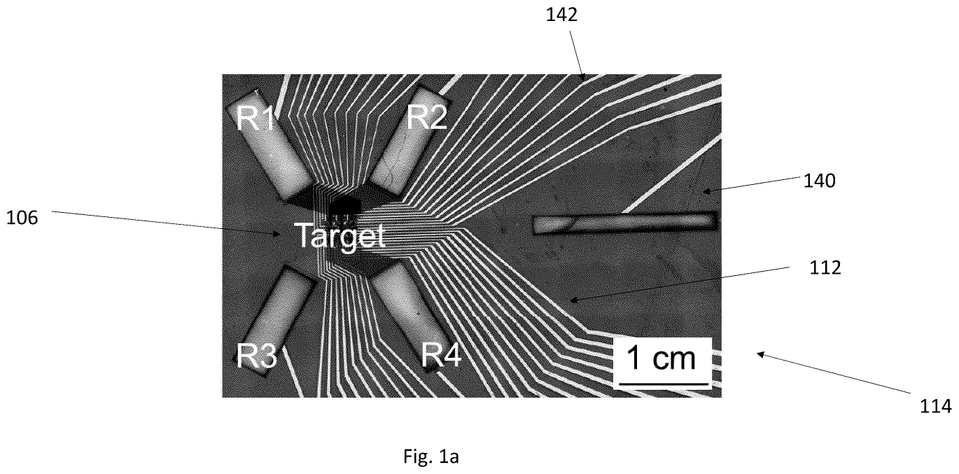

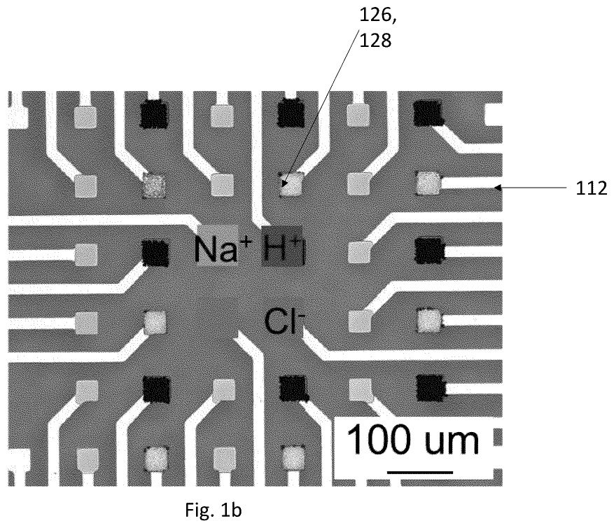

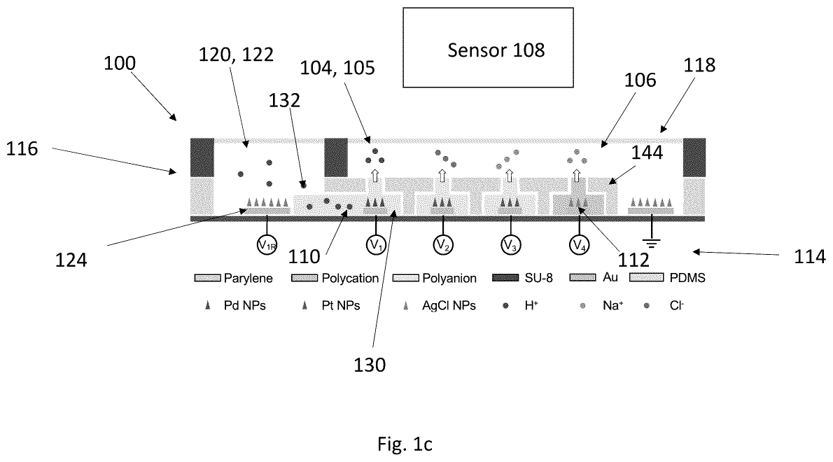

FIG. 1 shows the top view of the multi-ion pump, which comprises four independent reservoirs (R1, R2, R3, and R4) connected to one target. Each reservoir has one reference electrode and is connected to nine independent microelectrodes (3×3 array) in the target through an independent ion channel. FIG. 1(b) shows the mapping of the 36 microelectrodes in the target: every four microelectrodes form an array that is capable of delivering different ionic species. Here, we used three reservoirs to deliver H⁺, Na⁺, and ClVand named each individual type as H⁺pump, Na⁺pump, and Cl pump, respectively [FIG. 1(b)]. Nanoparticles were coated on the microelectrodes to increase the surface area and electrode capacitance. The microelectrodes of the H⁺pump were coated with Pd NPs, which are selective to H⁺ by forming PdH_{X}. ³⁰AgCl nanoparticles were coated on microelectrodes of the Cl pump because of its selectivity to Cl⁻. ³¹ For all the other general ionic species, platinum nanoparticles (Pt NPs) were deposited on the microelectrodes to increase the surface area and the capacitance for general delivery, such as Na⁺. The reference electrodes in the reservoir and auxiliary electrode were also AgClNP coated, which work as an electron-to-ion transducer. ³¹ On top of the microelectrodes, we deposited four independent ion channels that connected reservoirs and the target, which are ion conducting materials that allow ions to move following an external electric field. ³² Polymers with negative charges are selective to the cation and are used as ion channels for cation delivery, such as poly(2-acrylamido-2-methyl-1-propanesulfonic acid), while polymers with positive charges are selective to anion and are used as ion channels for anion delivery, such as poly (diallyldimethylammonium chloride). ³ Here, we chose Polyvinyl Alcohol: Polystyrene sulfonate (PVA:PSS) and PVA:Chitosan as ion channels. These ion channels are spin-coated and patterned by photolithography into two layers separated by parylene. Then, a SU8 photoresist was used to build the microfluidic channels to provide solutions for the reservoirs and target [FIG. 1(c)]. The microfluidic channels were sealed by microfluidic tape. FIG. 1(c) represents the side view of the multi-ion pump, showing the multilayer structure and the electrical circuits with H⁺reservoir as the representative.

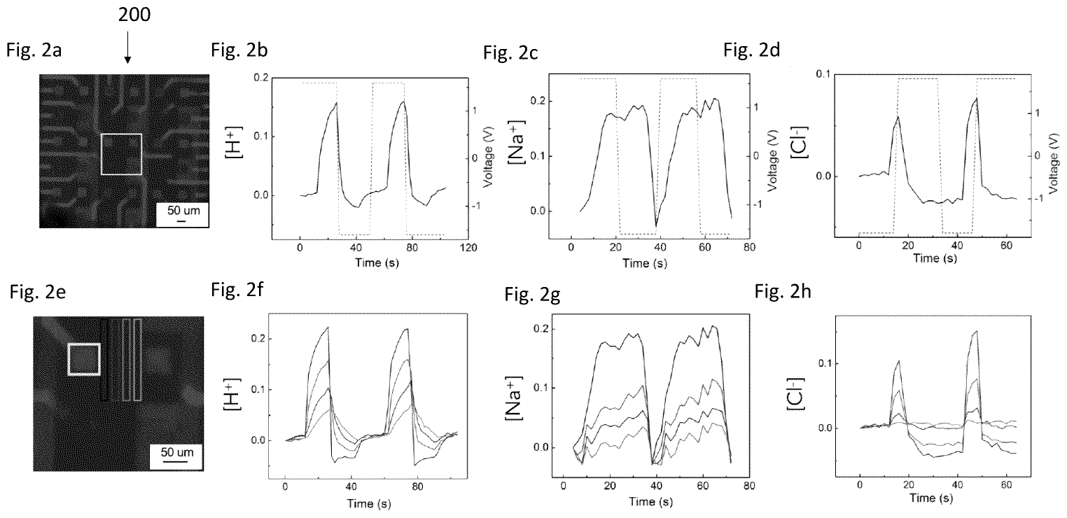

The results in FIG. 2 demonstrate control of the ion concentration of three ions: H⁺, Na⁺, and Cl⁻. In FIG. 2(b), first, V_{1R}=1.6 V, applied between the R1 in the H⁺reservoir and H⁺ microelectrodes, delivers H⁺ to the H⁺channel. Then, V₁=1.6 V delivers H⁺ from the microelectrodes into the target and increases [H⁺]in the area close to the microelectrodes as monitored by 5-(and-6)-Carboxy SNARF™-1. Next, reversing V₁ to be −1.6 V to absorb H⁺back into the H⁺channel, induced a [H⁺]decrease in the target.

FIG. 2(b) shows the [H⁺]change following V₁, indicating a quick and precise [H⁺]control by the voltage. [Na⁺]control was achieved by the same protocol as that of [H⁺]with 0.1MNaCl in the reservoir and CoroNa as the fluorescence indicator [FIG. 2(c)]because they are all cations. For [Cl], 0.1MNaCl was in the reservoir electrolyte and MQAE was used as a fluorescent indicator in the target electrolyte to monitor [Cl]in real time. Here, V_{4R}=−1.8 V between R4 in the Cl⁻ reservoir and Cl⁻ microelectrodes delivers Cl⁻ into the Cl channel. Then, V₄=−1.8 V delivers Cl to the target, increasing [Cl]of the area close to the microelectrode as measured.

Next, the reversal of the polarity of V₄ to 1.8 V induced absorption of Cl⁻ and reduced [Cl] in the target [FIG. 2(d)]. In addition to the temporal resolution, the high spatial resolution control of ion pumps is another critical advantage for ion/biomolecule delivery. In FIG. 2(e), when we applied voltage on one working microelectrode (highlighted in yellow square), the fluorescence intensity in the adjacent area (black, red, blue, and pink rectangular square) was measured to show the ion concentration gradient. Following the protocols that change [H⁺], [Na⁺], and [Cl⁻ ], we measured and plotted their fluorescence intensity in different areas [FIGS. 2(f)-2(h)]. The results show that while the monitored area gets further from the working microelectrodes, the ion concentration change by the voltage is diminished, indicating a precise control of spatial resolution.

Each microelectrode was designed to function independently. In addition, the demonstrated spatial ion fluxes also control by changing [H⁺]in different directions with two microelectrodes.

b. Control Loop

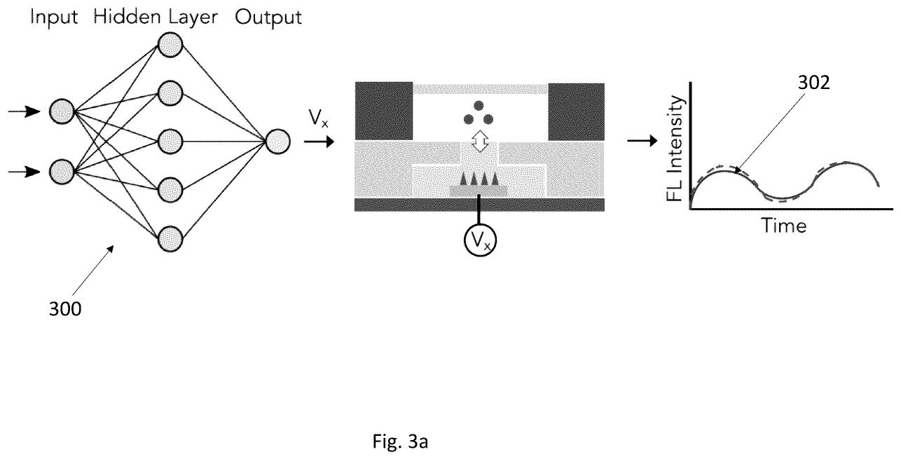

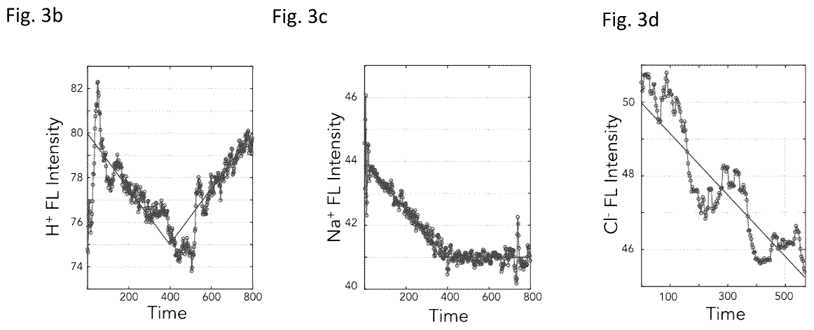

To apply ion delivery to biological processes is important to be able to closely control ion concentration toward a specific target value. ^{31,34,35} Traditional control methods are difficult for biological systems due to their complex dynamics and sensitivity to environmental changes. ³⁶ Therefore, integrating the machine-learningbased closed-loop control with the versatile multi-ion pump platform could introduce a powerful toolbox to further manipulate complex biological processes. Here, we successfully demonstrated the automated closed-loop control of ion fluxes by machine learning using the multi-ion pump. FIG. 3(a) shows the general architecture of the implemented online machine-learning-based controller designed for the multi-ion pump system. By the user choice, the ML-controller uses one of the four sub-controllers 34 for controlling the predefined ions. The blue line represents the reference signal, and the red line represents the system output (i.e., fluorescence intensity). In FIGS. 3(b)-3(d), we set different reference signals for different ions with blue lines. Then, we applied voltage through the controller to the corresponding microelectrodes and made the output to track the reference signals. The results show that we were able to control the ion fluxes of H⁺, Na⁺, and Cl⁻ following different patterns.

c. Device Fabrication

### (i) Process Flow

The multiple-ion pump platform was fabricated on a 4 in. borosilicate glass wafer using the following procedure.

Step 1: Au contacts and traces were patterned by a positive photoresist (S1813; Micro-Chem Corp.) and deposited by e-beam evaporation (10 nmTi, 100 nmAu). Acetone and IPA are used for liftoff.

Step 2: Subsequently, a S1813 photoresist is again used to selectively expose the Au contacts for nanoparticle electrodeposition.

Step 3: After electrodeposition, a 1.5 μm thick insulating layer of parylene-C was deposited (Specialty Coating Systems Labcoter 2 system) in the presence of an A174 adhesion promoter. The parylene was etched by an oxygen plasma with the regions over the electrodes and contact pads exposed and the rest protected by SPR220-4.5 or SPR220-7 (Micro-Chem Corp).

Step 4: Prior to the deposition of ion channels, (3-glycidyloxypropyl) trimethoxysilane (GOPS) was deposited on the wafer to promote adhesion of the polymer. 5% GOPS was dispersed in ethanol and spin coated at 1000 rpm for 30 s and then baked at 110° C. for 5 min.

A blend of 8 wt. % polyvinyl alcohol (PVA) with 2 wt. % polystyrene sulfonic acid (4:1 weight ratio) was thoroughly mixed by using a microwave and hotplate. The PVA:PSS solution was filtered by using a cellulose ester (MCE) syringe filter with 0.8 μm pore size and spin-coated on top of the wafer at 1500 rpm for 30 s and baked at 120 C for 2 h, yielding a film thickness of 2 μm. A positive photoresist Dow SPR2204.5 was spin-coated following the protocols of the manufacturer. The PVA:PSS film was etched with an oxygen plasma with the desired pattern defined with the SPR220-4.5 photoresist. Steps 5 and 6: A second 1.5 μm coating of parylene was then deposited with the same protocol as above to insulate and protect the PVA:PSS film by only exposing the 36 microelectrodes.

Step 7: To promote adhesion between the parylene and the next polymer layer, PVA:Chitosan, GOPS was again deposited using the aforementioned process prior to PVA:Chitosan patterning. Chitosan is dissolved in 1% acetic acid and mixed with 10 wt % PVA (1:2 weight ratio) thoroughly with the help of a microwave and hotplate. The PVA:Chitosan solution was filtered by using a cellulose ester (MCE) syringe filter with 0.8 μm pore size and spin-coated on top of the wafer at 1000 rpm for 5 s with 500 rpm/sramp and then 4500 rmp for 30 s with 1500 rpm/s and baked at 80 C for 2 h, yielding a film thickness of 2 μm. The PVA:Chitosan film was etched with an oxygen plasma with the desired pattern defined with a SPR220-4.5 photoresist.

Step 8: A third 1.5 μm coating of parylene was deposited with the same protocol as above to insulate and protect all the polymers from the subsequent SU8 deposition.

Step 9: To promote adhesion between the parylene and SU8 photoresist, GOPS was again deposited using the aforementioned process prior to SU8 patterning. SU8 3025 was spun onto the wafer at 500 rpm for 5 s with 100 rpm/sramp, 1000 rpm for 30 s with 300 rpm/sramp, and 3000 rpm for 1 s with 3000 rpm/sramp. The patterned 70 μm high SU8 photoresist formed the sidewalls of microfluidic channels for reservoir and target chambers.

Step 10: The third parylene insulation layer protecting the polymers was etched to expose the electrode contacts in the reservoir and target channels using the same process as the previous parylene etch.

Step 11: Finally, devices were diced from the wafers prior to sealing the microfluidics with single-sided microfluidic transparent diagnostic tape (3M 9964). Features in the tape layer were punched out with 1 and 2 mm diameter biopsy punches for exposing imaging area and fluidic inlets. The tapes were then aligned to SU8 features on the device and pressed to seal by hand. PDMS was also punched with the 1.5 mm diameter biopsy punches to provide support from the fluidic inlets.

### (ii) Electrodeposition

Electrodeposition is accomplished with a three-electrode configuration at room temperature with an Ag/AgCl pellet as a reference electrode and a platinum wire coil as a counter-electrode using an Autolab potentiostat. The following procedure yields the most repeatable and stable results among several plating procedures tested.

(iii) Pd NP Deposition

1 wt. % PdNO₃ solution was diluted from 10 wt. % PdNO₃ and used to electroplate the nanoparticles by applying a DC voltage of −0.3 V for 5 s. During this process, 10 μC charges are measured in the circuit, indicating that a single microelectrode with Pd NP coating theoretically has the capability of converting 10 μC electrons to H⁺ and vice versa.

### (iv) Pt NP Deposition

H₂PtCl₆ was dissolved in DI-water by 1:1 and used to electroplate the nanoparticles by applying a DC voltage of −0.06 V for 8 s. Here, we characterized the double layer capacitance by cyclic voltammetry and calculated the delivered charges to be 0.5 μC. It also shows that the ion-to-electron transducers (Pd/PdHx, Ag/AgCl) are much more efficient in ion delivery.

### (v) Ag/AgCl NP Deposition

10mMAgNO₃ in 0.1MKNO₃ was used to deposit Ag nanoparticles followed by chlorinating Ag nanoparticles into AgCl nanoparticles. For microelectrodes, −0.2 V was applied for 5 s. During this process, ³0.1 μC charges are measured in the circuit, indicating that a single microelectrode with the coating of Ag/AlCl NPs theoretically has the capability of converting 3.1 μC electrons to Cl⁻ and vice versa.

For a reference electrode and auxiliary electrode, −0.3 V was applied for 50 s. Then, we oxidize approximately AgNPs to AgCl NPs by applying a constant anodic current (100 μA) for 10 s on Ag NPs in 50mMKCl solution at room temperature. We observe a clear color change from silver white to dark gray, indicating the formation of AgCl NPs.

### (vi) Multi-Channel Potentiostat

The modular multi-channel potentiostat can operate multiple electrochemical devices or a single electrochemical device with more than one working electrode. The modular aspect allows the multi-channel potentiostat to scale from 8 to 64 channels by adding more stackable boards. The stackable board provides eight channels of circuits with the output range of ±4 V and the input range of ±1.65 μA. In addition, the modular multi-channel potentiostat also offers an external control mode where it allows for interfacing with external software, such as a machine-learning control algorithm. More detailed information of the multi-channel potentiostat can be found here. ³⁷

(vii) Fluorescence Probes

We used microscope-based real-time imaging over the microelectrodes to monitor the ion concentration change. We used 50 μM 5-(and-6)-Carboxy SNARF-1 (SNARF, ThermoFisher) dispensed in the 0.1M Tris buffer as a fluorescent indicator for H⁺, 50 μM CoroNa™ Green (CoroNa, ThermoFisher) dispensed in the 0.1M Tris buffer for Na⁺, and 100 μM [N-(ethoxycarbonylmethyl)-6methoxyquinolinium bromide] (MQAE, ThermoFisher) dispensed in the 0.1M Tris buffer for Cl⁻. The fluorescent probe solution was flowed into the target chamber via a sealed microfluidic channel. Thereafter, the device was monitored by using a BZ-X710 fluorescence microscope with 10× Nikon objective. Different filters are selected by the ion we are interested to image: TxRed (ex: 560/40 nm, em: 630/75 nm) for SNARF, GFP for CoroNa (ex: 502/30 nm, em: 520/36 nm, and DAPI (ex: 377/50 nm, em: 447/60 nm) for MQAE. Imaging data were collected every 2 s in real time. Data were analyzed using ImageJ software.

(viii) Machine-Learning-Based Closed-Loop Control

A machine-learning-based controller consists of four subcontrollers, one for each ion, performing in real-time and updating their parameters online. The ML-controller utilizes one of its four sub-controllers, one at a time. Each controller is based on the real-time adaptive machine-learning-based control methodology developed by the authors. ³⁴ More detailed information on the ML-based control algorithm could be found here. ³⁴

### References for the First Embodiment

The following references are incorporated by reference herein.
¹ H. Yuk, B. Lu, and X. Zhao, Chem. Soc. Rev. 48, 1642-1667 (2019).
² M. Jia and M. Rolandi, Adv. Healthcare Mater. 9, 1901372 (2020).
³ D. T. Simon, E. O. Gabrielsson, K. Tybrandt, and M. Berggren, Chem. Rev. 116, 13009-13041 (2016).
⁴ J. Selberg, M. Jia, and M. Rolandi, PLoS One 14, e0202713 (2019).
⁵ M. Jia, J. Kim, T. Nguyen, T. Duong, and M. Rolandi, Biopolymers 112, e23433 (2021).
⁶ A. D. Mickle, S. M. Won, K. N. Noh, J. Yoon, K. W. Meacham, Y. Xue, L. A. McIlvried, B. A. Copits, V. K. Samineni, K. E. Crawford, D. H. Kim, P. Srivastava, B. H. Kim, S. Min, Y. Shiuan, Y. Yun, M. A. Payne, J. Zhang, H. Jang, Y. Li, H. H. Lai, Y. Huang, S.-I. Park, R. W. Gereau, and J. A. Rogers, Nature 565, 361-365 (2019).
⁷ J. Kim, R. Ghaffari, and D.-H. Kim, Nat. Biomed. Eng. 1, 0049 (2017).
⁸ H. Dechiraju, M. Jia, L. Luo, and M. Rolandi, Adv. Sustainable Syst. 6, 2100173 (2021).
⁹ H. Chun and T. D. Chung, Annu. Rev. Anal. Chem. 8, 441-462 (2015).
¹⁰ M. Jia, L. Luo, and M. Rolandi, Macromol. Rapid Commun. 43, 2100687 (2022).
¹¹ J. Rivnay, S. Inal, A. Salleo, R. M. Owens, M. Berggren, and G. G. Malliaras, Nat. Rev. Mater. 3, 17086 (2018).
¹² J. Selberg, M. Jafari, J. Mathews, M. Jia, P. Pansodtee, H. Dechiraju, C. Wu, S. Cordero, A. Flora, N. Yonas, S. Jannetty, M. Diberardinis, M. Teodorescu, M. Levin, M. Gomez, and M. Rolandi, Adv. Intell. Syst. 2, 2000140 (2020).
¹³ Y. Deng, E. Josberger, J. Jin, A. F. Roudsari, B. A. Helms, C. Zhong, M. P. Anantram, and M. Rolandi, Sci. Rep. 3, 2481 (2013).
¹⁴ C. Zhong, Y. Deng, A. F. Roudsari, A. Kapetanovic, M. Anantram, and M. Rolandi, Nat. Commun. 2, 476 (2011).
¹⁵ E. E. Josberger, Y. Deng, W. Sun, R. Kautz, and M. Rolandi, Adv. Mater. 26, 4986-4990 (2014).
¹⁶ E. O. Gabrielsson, K. Tybrandt, and M. Berggren, Lab Chip 12, 2507-2513 (2012).
¹⁷ J. Zhu, Biomaterials 31, 4639-4656 (2010)
¹⁸ M. Mehrali, A. Thakur, C. P. Pennisi, S. Talebian, A. Arpanaei, M. Nikkhah, and A. Dolatshahi-Pirouz, Adv. Mater. 29, 1603612 (2017).
¹⁹ E. A. Kamoun, E.-R. S. Kenawy, and X. Chen, J. Adv. Res. 8, 217-233 (2017).
²⁰ M. Levin, Wiley Interdiscip. Rev.: Syst. Biol. Med. 5, 657-676 (2013).
²¹ M. Chesler, Physiol. Rev. 83, 1183-1221 (2003).
²² D. Arosio and G. M. Ratto, Front. Cell. Neurosci. 8, 258 (2014).
²³ A. Williamson, J. Rivnay, L. Kergoat, A. Jonsson, S. Inal, I. Uguz, M. Ferro, A. Ivanov, T. A. Sjöström, D. T. Simon, M. Berggren, G. G. Malliaras, and C. Bernard, Adv. Mater. 27, 3138-3144 (2015).
²⁴ D. T. Simon, S. Kurup, K. C. Larsson, R. Hori, K. Tybrandt, M. Goiny, E. W. H. Jager, M. Berggren, B. Canlon, and A. Richter-Dahlfors, Nat. Mater. 8, 742-746 (2009).
²⁵ A. Jonsson, Z. Song, D. Nilsson, B. A. Meyerson, D. T. Simon, B. Linderoth, and M. Berggren, Sci. Adv. 1, e1500039 (2015).
²⁶ D. J. Poxson, M. Karady, R. Gabrielsson, A. Y. Alkattan, A. Gustavsson, S. M. Doyle, S. Robert, K. Ljung, M. Grebe, D. T. Simon, and M. Berggren, Proc. Natl. Acad. Sci. U.S.A 114, 4597-4602 (2017).
²⁷ D. S. Adams, S. G. M. Uzel, J. Akagi, D. Wlodkowic, V. Andreeva, P. C. Yelick, A. Devitt-Lee, J.-F. Pare, and M. Levin, J. Physiol. 594, 3245-3270 (2016).
²⁸ L. N. Vandenberg, R. D. Morrie, and D. S. Adams, Dev. Dyn. 240, 1889-1904 (2011).
²⁹ A. Jonsson, T. A. Sjöström, K. Tybrandt, M. Berggren, and D. T. Simon, Sci. Adv. 2, e1601340 (2016).
³⁰ M. Jia, S. Ray, R. Breault, and M. Rolandi, APL Mater. 8, 120704 (2020).
³¹ M. Jia, H. Dechiruji, J. Selberg, P. Pansodtee, J. Mathews, C. Wu, M. Levin, M. Teodorescu, and M. Rolandi, APL Mater. 8, 091106 (2020).
³² B. D. Paulsen, K. Tybrandt, E. Stavrinidou, and J. Rivnay, Nat. Mater. 19, 13-26 (2020).
³³ T. Arbring Sjöström, M. Berggren, E. O. Gabrielsson, P. Janson, D. J. Poxson, M. Seitanidou, and D. T. Simon, Adv. Mater. Technol. 3, 1700360 (2018). ³⁴ M. Jafari, G. Marquez, J. Selberg, M. Jia, H. Dechiraju, P. Pansodtee, M. Teodorescu, M. Rolandi, and M. Gomez, IEEE Control Syst. Lett. 5, 1133 (2020).
³⁵ B. Hosseini Jafari, K. Zlobina, G. Marquez, M. Jafari, J. Selberg, M. Jia, M. Rolandi, and M. Gomez, J. R. Soc., Interface 18, 20210497 (2021).
³⁶ J. Selberg, M. Jafari, C. Bradley, M. Gomez, and M. Rolandi, APL Mater. 8, 120904 (2020).
³⁷ V. T. Ly, P. V. Baudin, P. Pansodtee, E. A. Jung, K. Voitiuk, Y. M. Rosen, H. Rankin Willsey, G. L. Mantalas, S. T. Seiler, J. A. Selberg, S. A. Cordero, J. M. Ross, M. Rolandi, A. A. Pollen, T. J. Nowakowski, D. Haussler, M. A. Mostajo-Radji, and S. R. Salama, Communications Biology 4, 1-4 (2021).

### Second Embodiment: Programmable Delivery of Therapy (e.g., Fluoxetine) Via Wearable Bioelectronics Accelerates Wound Healing In Vivo

### (i) Device Architecture

The second embodiment describes a programmable bioelectronic bandage capable of delivering the drug fluoxetine (brand name: Prozac) as a personalized treatment regimen to accelerate wound healing in mice. Studies have demonstrated that topical administration of fluoxetine improves diabetic and non-diabetic wound contraction and closure [12], decreases wound inflammation, and minimizes infection [13].

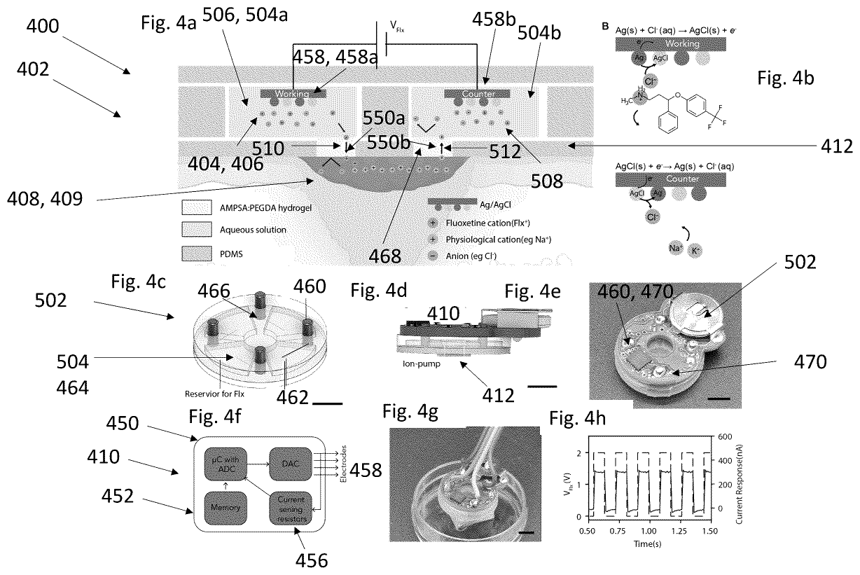

FIG. 4 showcases the design and characterization of the wearable bioelectronic bandages. (A) The ion pump's working principle is illustrated, where the reservoirs are filled with 0.01 M fluoxetine hydrochloride (FlxCl) solution. Under positive V_{Flx}, the Flx⁺ cations migrate from the reservoir to the wound under electrophoretic force and exchange with physiological cations. An ion selective AMPSA:PEGDA polyanion hydrogel (light yellow) is negatively charged, acting as a filter that blocks anions such as Cl⁻, while allowing cations such as Flx⁺ to pass. (B) The electrochemical reaction for the ion pump electrodes is depicted happening at the opposite direction simultaneously. The Ag on the working electrode (gray) becomes oxidized under the electrochemical reaction, absorbs a Cl⁻ from the Flx·HCl solution, releases an electron, and becomes AgCl. This reaction releases an electron to the external circuit and leaves a free Flx⁺ cation. The Flx⁺ then migrates to the wound under electrophoretic force. The cathode reaction consumes an electron and releases a free Cl⁻, which can balance with the incoming cation. (C) A CAD model of the ion pump, made from PDMS. The ion pump has four reservoirs for fluoxetine solution, where electrode wires made from Ag and AgCl are inserted and connected to the external circuit through contact pins made of steel. The scale bar is 5 mm. (D) CAD models of the wearable bioelectronic bandage device assembly, with the controller module on top and the ion pump on the bottom, are displayed. The scale bar is 5 mm. (E) A camera photo of the wearable bioelectronic bandage is presented, and the scale bar is 5 mm. (F) The circuit diagram of the controller module is provided. It includes a microcontroller with a built-in clock and analog-to-digital converter (ADC), a memory chip, a digital-to-analog converter (DAC), and resistors for sensing current. (G) A camera photo of the ex-vivo test setup for the wearable bioelectronic bandage while the device is in contact with the saline solution inside a PDMS well is shown, and the scale bar is 5 mm. (H) The plot of the device's current response to a 2V (peak to peak) square wave applied with a potentiostat is presented, indicating high temporal resolution.

### (ii) In Vivo Implementation and Testing

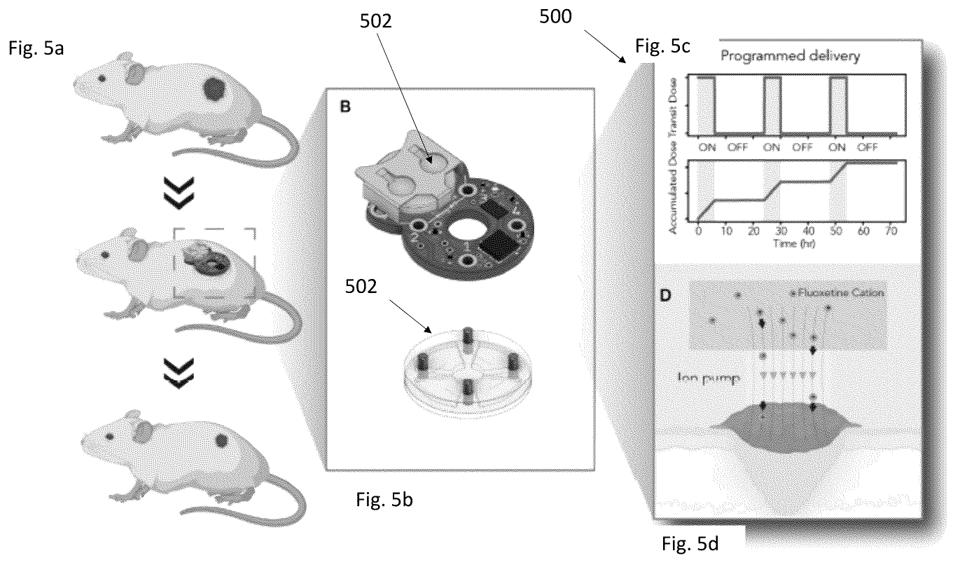

FIG. 5 illustrates the bioelectronic bandage implemented as wearable device that consists of two modules: an ion pump drug delivery module and a battery-powered controller module. The controller module is responsible for translating a pre-programmed delivery profile into a sequence of voltage signals (FIG. 5C), which activate the ion pump to deliver fluoxetine to the wound (FIG. 5D).

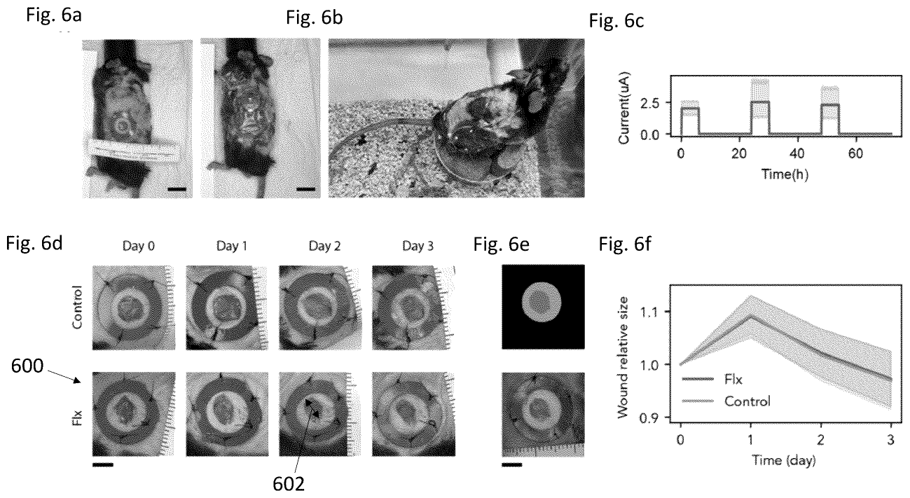

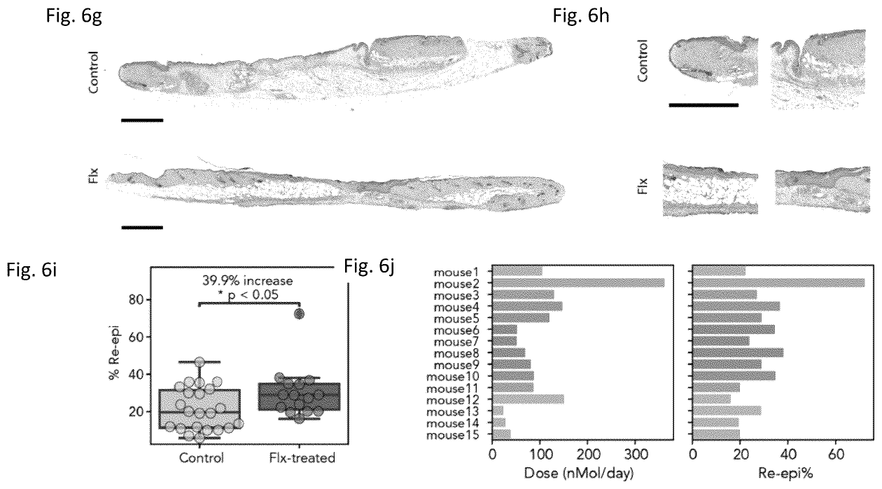

FIG. 6 illustrates testing of the efficacy of the bioelectronic bandage in vivo on a mouse wound model. We created a 6 mm punch biopsy wound held open by a silicone splint ring to minimize wound contraction (FIG. 6A). Wound contraction is a major wound healing process in mice but not humans. It confounds the histological analysis of the wound reponse to therapy, particularly re-epithelialization [12b, 13]. A gas-permeable, transparent adhesive dressing, Tegaderm™, fixes the bioelectronic bandage to the mouse (FIG. 6A, right) so that the mouse can move around its cage without interference (FIG. 6B). This is possible because the lightweight bandage (2.5 g) represents less than 10% of the mouse weight. For this test, we programmed the bandage to deliver Flx⁺ for three days for six hours per day (FIG. 6C). From our estimates, this program should deliver approximately 100 nMol of fluoxetine per wound per day—a Flx⁺ dose that has been shown to improve healing in a mouse wound model when topically applied to the wound bed [12b, 13]. A blinking LED on the bioelectronic bandage indicated that the program was running as desired.

We utilized a machine-learning algorithm reported [16] to analyze images of wounds and evaluate their condition and healing progression (FIG. 6D). The algorithm automatically marked the wound area and analyzed the wound size (FIG. 6E). Both the control and fluoxetine-treated wounds exhibited a similar trend of a slight increase in size at day 1, followed by a decrease on subsequent days, which aligns with the expected size change during wound healing (FIG. 6F). To further investigate healing, the wounds were fully excised at the end of the experiment, fixed, and stained with hematoxylin and eosin (H&E) to assess wound re-epithelialization (FIG. 6G-H). Re-epithelialization, contributed to by both keratinocyte proliferation and migration, is required for wound closure [17]. Treatment of wounds with Flx⁺ delivered from the bioelectronic bandage resulted in a 39.9% (P<0.05) increase in re-epithelialization compared to control (FIG. 6I), indicating a significant improvement in early-stage healing. Across 15 mice, the bioelectronic bandage consistently delivered the desired dose with some variability (FIG. 6J, left). This variability in delivered dose can be easily amended with current control using closed-loop control algorithms, as we have previously demonstrated with other ion pumps [18]. This variability allowed us to qualitatively correlate the dose of Flx⁺ and wound healing as measured by the percentage of re-epithelization (FIG. 6J, right). For example, reepithelization in mouse 2, which received >300 nMol per day Flx⁺, was much faster than in mouse 15, which received a 10× smaller dose. This observation is consistent with the direct topical application of fluoxetine to murine skin wounds [12b, 13]. While the correlation between fluoxetine dose and re-epithelization is strong (R²=0.58), it is not statistically significant due to the small sample size.

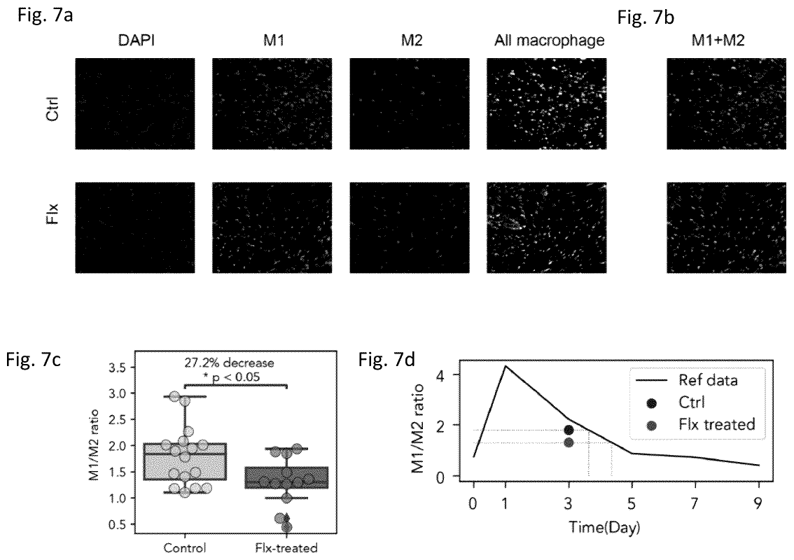

After analyzing the promising re-epithelization data, we delved deeper into another crucial indicator of wound healing: the M1/M2 macrophage ratio (FIG. 7) [19]. Macrophages play a pivotal role in the immune response and are crucially involved in the healing and regeneration of wounds. Although macrophages are phenotypically heterogeneous over a continuum, a simplified classification based on their polarized functions during the different stages of wound repair [19] defines the M1 subtype that carries out pro-inflammatory activities [20] and the M2 subtype that is anti-inflammatory [21] and promotes tissue repair [22]. Using immunohistochemistry to identify the subtypes based on the expression of recognized markers, we found M1 macrophages significantly infiltrated the center of the control wound three days after injury, while there was no change observed in the fluoxetine treated wound (FIG. 7A). Meanwhile, the number of M2-like macrophages increased in the center of the fluoxetine-treated wound, as depicted in FIG. 7A. At day 3 the M1/M2 ratio was noticeably reduced following treatment, as shown in the overlay of M1 and M2 cells (FIG. 7B). Fluoxetine treatment additionally reduced the M1/M2 ratio on day 3 by 27.2% compared to control (P<0.05) (FIG. 7C) indicating a lower number of the M1 proinflammatory macrophages compared to the M2 pro-reparative macrophages. This M1/M2 ratio decrease suggests a shorter inflammatory phase with a more rapid progression towards the reparative phase of healing, consistent with the noted improvement in wound reepithelization.

To further investigate this effect, we examined the M1/M2 ratio change in the context of a continuous curve over the healing process. Using time series data of M1 and M2 cells in mouse incision wounds obtained from published studies [23], we plotted the M1/M2 ratio's dependence on time (FIG. 7D). Though the comparison of the published data generated from incisional wounds [23] to the current data generated from excisional wounds may be an imperfect approach, nevertheless, plotting M1/M2 ratio along with published data enables us to estimate the wound progression. The M1/M2 ratio of both fluoxetine-treated and control wounds on day 3 of this experiment are within the same order of magnitude (blue and red dots in FIG. 7D) as the time-series obtained from published data (black line in FIG. 7D). However, because the curve is non-monotonic, there are two periods that might correspond to the M1/M2 value obtained in this study—one is day 0, and another is days 3-5. After taking into account other wound indicators, such as the onset of re-epithelialization, the presence of macrophages, and diminishing wound size, we conclude that our day 3 data correspond to the day 3-5 part of the curve, i.e. M1/M2 ratio is monotonically decreasing in that time period. Therefore, a lower M1/M2 ratio indicates that the wound has entered a later healing stage.

Previous studies reported that the M1-M2 transition is critical for the resolution of inflammation and for promoting tissue repair [24]. Since the fluoxetine-treated wounds showed increased re-epithelialization and decreased M1/M2 ratio, we conclude that the wearable bioelectronic bandage's fluoxetine treatment accelerated the wound-healing process. This finding is consistent with earlier studies that directly applied fluoxetine to the wound bed [12b, 13]. Although fluoxetine is a selective serotonin reuptake inhibitor (SSRI) primarily used as a systemically administered drug for treating depression, recent studies have revealed that SSRIs may affect various types of cells involved in cutaneous wound healing, such as keratinocytes, fibroblasts, endothelial, and immune cells [25], and modify their migration, differentiation, and function. Moreover, since the wearable bioelectronic bandage's delivery is localized to the wound, there is little to no systemic accumulation of fluoxetine or impact on the serotonin metabolism, reducing unwanted side effects [26].

(iii) Fabrication of Ion Pump

To fabricate the ion pump for the wearable bioelectronic bandage, AutoCAD software was used to design 3D-printed, two-part molds. The molds were filled with Polydimethylsiloxane (PDMS) and baked at 60° C. for 48 hours. The resulting PDMS parts were then removed from the molds and cleaned with Isopropyl Alcohol (IPA) and water, followed by nitrogen (N₂) drying to ensure no debris remained on the PDMS layers. The top layer of PDMS contained four reservoirs, designed to hold fluoxetine solutions of specific concentrations, and four capillary tubes filled with hydrogels for fluoxetine delivery. The bottom PDMS part acted as a lid, covering the reservoirs and featuring a 0.5 mm tall notch to ensure contact with the wound bed below the skin. Silver (Ag) and silversilver chloride (Ag/AgCl) wires with a diameter of 0.1 mm were inserted inside each reservoir. The top and bottom layers were bonded together through oxygen (O₂) plasma treatment, which oxidizes the polymer surface and changes the CH₃ groups on the PDMS surface to OH groups. The oxidized surfaces were bonded together using custom aluminum pieces. The PDMS surface was coated with Parylene to increase the media lifetime on the PDMS reservoirs. Hydrogel-filled capillaries, which act as the ion exchange membrane for the ion pump, were fabricated using a previously optimized and reported method [27]. The hydrogel recipe in this study consisted of a 1M concentration of 2-acrylamido-2-methyl-1propanesulfonic acid (AMPSA), 0.4M concentration of polyethylene glycol diacrylate (AMPSA), and 0.05M concentration of photoinitiator (I2959). 100 mm of silica tubing with an inner diameter of 100 μm and an outer diameter of 375 μm were etched with NaOH and then treated with silane A174 to prevent hydrogel expansion. The hydrogel was crosslinked with five minutes of 365 nmUV treatment at a power density of 8 mW cm⁻². After UV curing, the capillary tubes were segmented into 5 mm segments and loaded by immersing them in a 0.01M fluoxetine solution for at least 4 hours before use. Finally, the capillaries filled with hydrogel were inserted into each reservoir to complete the fabrication of the ion pump.

### (iv) Design and Fabrication of Controller Module

The controller module for drug delivery comprises a programmable PCB with electronic components used for actuation and sensing. The PCB was designed using Autodesk EAGLE and contains a microcontroller with a built-in ADC, a memory chip, a DAC, and resistors for sensing current (FIG. 1F). Prior to the experiment, programs were flashed into the onboard microcontroller, and actuation commences automatically once the battery is inserted. During program execution, the microcontroller sends I 2C commands to the DAC to apply the appropriate voltages to the electrodes of the ion pump. As a result, current flows through the resistors, generating voltages that can be read by either the ADC on the microcontroller or by external probes.

### (v) Assembling of the Wearable Bioelectronic Bandage

To integrate the two modules of the wearable bioelectronic bandage, steel pins were inserted into four holes on the PDMS layer of the ion pump. The bottom of each pin was coated with silver paste to establish electrical connections between the pin and Ag or Ag/AgCl electrodes. After assembling the wearable bioelectronic bandage, the pins were soldered to the PCB. Sterilized fluoxetine hydrochloride solutions (0.01M) were then prepared by dissolving the drug in sterilized water, adjusting the pH to 6 to allow the fluoxetine to protonate, filtering through 0.2 μm filters, and injecting the sterilized fluoxetine solutions into each reservoir.

### References for Second Embodiment

The following references are incorporated by reference herein
[1] L. L. O. B. e. al., Goodman & Gilman's the pharmacological basis of therapeutics, McGraw-Hill, New York 2006.
[2] a) J. V. Natarajan, C. Nugraha, X. W. Ng, S. Venkatraman, J Control Release 2014, 193, 122, https://doi.org/10.1016/j.jconrel.2014.05.029, b) T. Thambi, Y. Li, D. S. Lee J Control Release 2017, 267, 57, https://doi.org/10.1016/j.jconrel.2017.08.066; c) Z. Hemmatian, E. Jalilian, S. Lee, X. Strakosas, A. Khademhosseini, A. Almutairi, S. R. Shin, M. Rolandi, ACS Appl Mater Interfaces 2018, 10 (26), 21782, https://doi.org/10.1021/acsami.8b02724.
[3] a) H. Joo, Y. Lee, J. Kim, J. S. Yoo, S. Yoo, S. Kim, A. K. Arya, S. H. Choi, N. Lu, H. S. Lee, S. T. Lee, D. H. Kim, SciAdv 2021, 7 (1), eabd4639, https://doi.org/10.1126/sciadv.abd4639; b) B. H. McAdams, A. A. Rizvi, J Clin Med 2016, 5 (1), 5, https://doi.org/10.3390/jcm5010005.
[4] a) X. Strakosas, M. Seitanidou, K. Tybrandt, M. Berggren, D. T. Simon, Sci Adv 2021, 7 (5), eabd8738, https://doi.org/10.1126/sciadv.abd8738; b) D. T. Simon, E. W. H. Jager, K. Tybrandt, K. C. Larsson, S. Kurup, A. Richter-Dahlfors, B. Berggren, An organic electronic ion pump to regulate intracellular signaling at high spatiotemporal resolution. In
TRANSDUCERS 2009-2009 International Solid-State Sensors, Actuators and Microsystems Conference, 2009; pp 1790-1793; c) M. P. Jia, H. Dechiruji, J. Selberg, P. Pansodtee, J. Mathews, C. X. Wu, M. Levin, M. Teodorescu, M. Rolandi, Apl Mater 2020, 8 (9), 091106, https://doi.org/Artn 091106 10.1063/5.0013867; d) M. P. Jia, M. Jafari, P. Pansodtee, M. Teodorescu, M. Gomez, M. Rolandi, Apl Mater 2022, 10 (4), 041112, https://doi.org/Artn 041112 10.1063/5.0084570.
[5] a) C. M. Proctor, A. Slezia, A. Kaszas, A. Ghestem, I. Del Agua, A. M. Pappa, C. Bernard, A. Williamson, G. G. Malliaras, Sci Adv 2018, 4 (8), eaaul291, https://doi.org/10.1126/sciadv.aau1291, b) I. Uguz, C. M. Proctor, V. F. Curto, A. M. Pappa, M. J. Donahue, M. Ferro, R. M. Owens, D. Khodagholy, S. Inal, G. G. Malliaras, Adv Mater 2017, 29 (27), 1701217, https://doi.org/10.1002/adma.201701217; c) A. Jonsson, T. A. Sjostrom, K. Tybrandt, M. Berggren, D. T. Simon, Sci Adv 2016, 2 (11), e1601340, https://doi.org/10.1126/sciadv. 1601340.
[6] M. Zhao, M. Rolandi, R. R. Isseroff, Cold Spring Harb Perspect Biol 2022, 14 (10), a041236, https://doi.org/10.1101/cshperspect.a041236.
[7] a) R. W. Tarnuzzer, G. S. Schultz, Wound Repair Regen 1996, 4 (3), 321, https://doi.org/10.1046/j.1524-475X.1996.40307.x; b) D. R. Yager, R. A. Kulina, L. A. Gilman, The International Journal of Lower Extremity Wounds 2007, 6 (4), 262, https://doi.org/10.1177/1534734607307035.
[8] a) D. Y. Matar, B. Ng, O. Darwish, M. Wu, D. P. Orgill, A. C. Panayi, Adv Wound Care (New Rochelle) 2023, 12 (5), 269, https://doi.org/10.1089/wound.2021.0126; b) P. Martin, Science 1997, 276 (5309), 75, https://doi.org/10.1126/science.276.5309.75; c) S. Willenborg, L. Injarabian, S. A. Eming, Cold Spring Harb Perspect Biol 2022, 14 (12), a041216, https://doi.org/10.1101/cshperspect.a041216.
[9] V. Falanga, R. R. Isseroff, A. M. Soulika, M. Romanelli, D. Margolis, S. Kapp, M. Granick, K. Harding, Nat Rev Dis Primers 2022, 8 (1), 50, https://doi.org/10.1038/s41572022-00377-3.
[10] a) Y. Jiang, A. A. Trotsyuk, S. Niu, D. Henn, K. Chen, C. C. Shih, M. R. Larson, A. M. Mermin-Bunnell, S. Mittal, J. C. Lai, A. Saberi, E. Beard, S. Jing, D. Zhong, S. R. Steele, K. Sun, T. Jain, E. Zhao, C. R. Neimeth, W. G. Viana, J. Tang, D. Sivaraj, J. Padmanabhan, M. Rodrigues, D. P. Perrault, A. Chattopadhyay, Z. N. Maan, M. C. Leeolou, C. A. Bonham, S. H. Kwon, H. C. Kussie, K. S. Fischer, G. Gurusankar, K. Liang, K. Zhang, R. Nag, M. P. Snyder, M. Januszyk, G. C. Gurtner, Z. Bao, Nat Biotechnol 2022, 1, https://doi.org/10.1038/s41587-022-01528-3; b) C. Wang, X. Jiang, H. J. Kim, S. Zhang, X. Zhou, Y. Chen, H. Ling, Y. Xue, Z. Chen, M. Qu, L. Ren, J. Zhu, A. Libanori, Y. Zhu, H. Kang, S. Ahadian, M. R. Dokmeci, P. Servati, X. He, Z. Gu, W. Sun, A. Khademhosseini, Biomaterials 2022, 285, 121479, https://doi.org/10.1016/j.biomaterials.2022.121479.
[11] P. Mostafalu, G. Kiaee, G. Giatsidis, A. Khalilpour, M. Nabavinia, M. R. Dokmeci, S. Sonkusale, D. P. Orgill, A. Tamayol, A. Khademhosseini, Adv Funct Mater 2017, 27 (41), 1702399, https://doi.org/ARTN 1702399 10.1002/adfm. 201702399.
[12] a) R. M. Farahani, K. Sadr, J. S. Rad, M. Mesgari, Adv Skin Wound Care 2007, (3), 157, https://doi.org/10.1097/01.ASW.0000262710.59293.6b; b) C. M. Nguyen, D. M. Tartar, M. D. Bagood, M. So, A. V. Nguyen, A. Gallegos, D. Fregoso, J. Serrano, D. Nguyen, D. Degovics, A. Adams, B. Harouni, J. J. Fuentes, M. G. Gareau, R. W. Crawford, A. M.
Soulika, R. R. Isseroff, Diabetes 2019, 68 (7), 1499, https://doi.org/10.2337/db18-1146; c) N. A. Alhakamy, G. Caruso, A. Privitera, O. A. A. Ahmed, U. A. Fahmy, S. Md, G. A. Mohamed, S. R. M. Ibrahim, B. G. Eid, A. B. Abdel-Naim, F. Caraci, Pharmaceutics 2022, 14 (6), 1133, https://doi.org/10.3390/pharmaceutics14061133.
[13] D. J. Yoon, C. Nguyen, M. D. Bagood, D. R. Fregoso, H. Y. Yang, A. I. M. Lopez, R. W. Crawford, J. Tran, R. R. Isseroff, J Invest Dermatol 2021, 141 (6), 1608, https://doi.org/10.1016/j.jid.2020.11.016.
[14] N. J. Trengove, S. R. Langton, M. C. Stacey, Wound Repair Regen 1996, 4 (2), 234, https://doi.org/10.1046/j 1524-47SX 1996.40211.x.
[15] M. Seitanidou, J. F. Franco-Gonzalez, T. A. Sjöström, I. Zozoulenko, M. Berggren, D. T. Simon, J Phys Chem B 2017, 121 (30), 7284, https://doi.org/10.1021/acs.jpcb.7605218.
[16] H. Carrion, M. Jafari, M. D. Bagood, H. Y. Yang, R. R. Isseroff, M. Gomez, PLoS Comput Biol 2022, 18 (3), e1009852, https://doi.org/10.1371/journal.pcbi. 1009852.
[17] a) H. Y. Yang, F. Fierro, D. J. Yoon, A. Gallegos, S. L. Osborn, A. V. Nguyen, T. R. Peavy, W. Ferrier, L. Talken, B. W. Ma, K. G. Galang, A. Medina Lopez, D. R. Fregoso, H. Stewart, E. A. Kurzrock, A. M. Soulika, J. A. Nolta, R. R. Isseroff, J Biomed Mater Res B Appl Biomater 2022, 110 (7), 1615, https://doi.org/10.1002/jbm.b.35022; b)H. Y. Yang, F. Fierro, M. So, D. J. Yoon, A. V. Nguyen, A. Gallegos, M. D. Bagood, T. Rojo-Castro, A. Alex, H. Stewart, M. Chigbrow, M. R. Dasu, T. R. Peavy, A. M. Soulika, J. A. Nolta, R. R. Isseroff, Stem Cells Transl Med 2020, 9 (11), 1353, https://doi.org/10.1002/sctm.19-0380; c) K. Ueno, S. Saika, Y. Okada, H. Iwanishi, K. Suzuki, G. Yamada, S. Asamura, Exp Anim 2022, https://doi.org/10.1538/expanim.22-0124; d) R. D. Galiano, J. t. Michaels, M. Dobryansky, J. P. Levine, G. C. Gurtner, Wound Repair Regen 2004, 12 (4), 485, https.//doi.org/10.1111/j.1067-1927.2004.12404.x; e) S. Erratico, M. Belicchi, M. Meregalli, D. Di Silvestre, L. Tripodi, A. De Palma, R. Jones, E. Ferrari, L. Porretti, E. Trombetta, G. R. Merlo, P. Mauri, Y. Torrente, Cell Mol Life Sci 2022,79 (5), 259, https://doi.org/10.1007/s00018-022-04284-4.
[18] a) J. Selberg, M. Jafari, C. Bradley, M. Gomez, M. Rolandi, Apl Mater 2020, 8 (12), 120904, https://doi.org/Artn 120904 10.1063/5.0027226; b) H. Dechiraju, J. Selberg, M. P. Jia, P. Pansodtee, H. P. Li, H. C. Hsieh, C. Hernandez, N. Asefifeyzabadi, T. Nguyen, P. Baniya, G. Marquez, C. Rasmussen-Ivey, C. Bradley, M. Teodorescu, M. Gomez, M. Levin, M. Rolandi, Aip Adv 2022, 12 (12), 125205, https://doi.org/Artn 125205 10.1063/5.0129134; c) J. Selberg, M. Jafari, J. Mathews, M. P. Jia, P. Pansodtee, H. Dechiraju, C. X. Wu, S. Cordero, A. Flora, N. Yonas, S. Jannetty, M. Diberardinis, M. Teodorescu, M. Levin, M. Gomez, M. Rolandi, Adv Intell Syst-Ger 2020, 2(12), 2000140, https://doi.org/ARTN 2000140 10.1002/aisy.202000140.
[19] a) D. M. Mosser, J. P. Edwards, Nat Rev Immunol 2008, 8 (12), 958, https://doi.org/10 1038/nri2448; b) T. Lucas, A. Waisman, R. Ranjan, J. Roes, T. Krieg, W. Muller, A. Roers, S. A. Eming, J Immunol 2010, 184 (7), 3964, https://doi.org/10.4049/jimmunol.0903356.
[20] D. C. Dale, L. Boxer, W. C. Liles, Blood 2008, 112 (4), 935, https://doi org/10.1182/blood-2007-12-077917.
[21] a) M. M. Hunter, A. Wang, K. S. Parhar, M. J. Johnston, N. Van Rooijen, P. L. Beck, D. M. McKay, Gastroenterology 2010, 138 (4), 1395,
https://doi.org/10.1053/j.gastro.2009.12.041; b) S. K. Brancato, J. E. Albina, Am J Pathol
2011, 178 (1), 19, https://doi.org/10.1016/j.ajpath.2010.08.003.
[22] S. Gordon, Nat Rev Immunol 2003, 3 (1), 23, https://doi.org/10.1038/nri978.
[23] Y. Du, P. Ren, Q. Wang, S. K. Jiang, M. Zhang, J. Y. Li, L. L. Wang, D. W. Guan, J Inflamm (Land) 2018, 15, 25, https://doi.org/10.1186/sl295-018-0201-z.
[24] N. X. Landen, D. Li, M. Stahle, Cell Mol Life Sci 2016, 73 (20), 3361, https://doi.org/10.1007/sW0018-016-2268-0.
[25] a) A. Slominski, J. Wortmman, D. J. Tobin, FASEB J 2005, 19 (2), 176,
https://doi.org/10.1096/fj. 04-2079rev; b) R. Arreola, E. Becerril-Villanueva, C. Cruz-Fuentes, M. A. Velasco-Velazquez, M. E. Garces-Alvarez, G. Hurtado-Alvarado, S.

Quintero-Fabian, L. Pavon, J Immunol Res 2015, 2015, 354957,

https://doi.org/10.1155/2015/354957: c) M. de Las Casas-Engel, A. L. Corbi, Adv Exp Med Biol 2014, 824, 89, https://doi.org/10.1007/9783-319-07320-0_9; d) D. A. Mann, F. Oakley, Biochim Biophys Acta 2013, 1832(7), 905,
https://doi.org/10.1016/j.bbadis.2012.0.009; e) K. Nordlind, E. C. Azmitia, A. Slominski, Exp Dermatol 2008, 17 (4), 301, https://doi.org/10.1111/j.1600-0625.2007.00670.x.
[26] a) M. Koelch, A. K. Pfalzer, K. Kliegl, S. Rothenhöfer, A. G. Ludolph, J. M. Fegert, R. Burger, C. Mehler-Wex, J. Stingi, R. Taurines, K. Egberts, M. Gerlach,
Pharmacopsychiatry 2012, 45 (2), 72; b) I. Inkielewicz-Stepniak, Pharmacological reports: PR 2011, 63 (2), 441; c) M. S. Golub, C. E. Hogrefe, Psychopharmacology 2014, 231 (20), 4041.
[27] M. Jia, L. Luo, M. Rolandi, Macromolecular Rapid Communications 2022, 43 (6), 2100687, https://doi.org/10.1002/marc. 202100687.
[28] A. Gallegos, R. R. Isseroff, Methods X 2022, 9, 101624, https://doi.org/10.1016/j.mex.2022.101024.

### System Embodiment: Intelligent Wound Care Management System

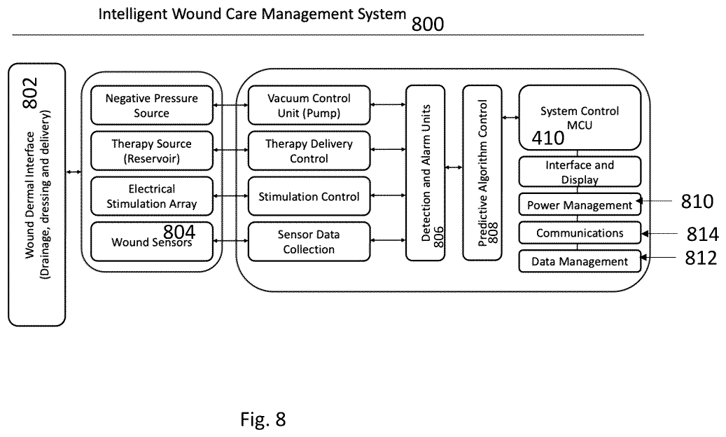

FIG. 8 illustrates an intelligent wound care management system comprising the wearable device described herein. The system comprises a wound dermal interface for attaching the device to the treatment site; sensors coupled to the treatment site positioned for measuring the dose and/or a healing state of the treatment site and outputting healing data in response thereto; and an alarm system coupled to the sensors, the alarm system comprising one or more processors configured for determining whether the healing data is within an acceptable range for the treatment and outputting an alarm signal indicating whether the healing data is within the acceptable range or not.

The system further comprises a computer comprising one or more processors configured for executing a predictive algorithm to determine scheduling of the control voltages applied to the electrodes in response to the alarm signal and outputting prediction data.

The system further comprises a power management system comprising a power source coupled to the device, for distributing power to the device; a data management system configured for storing the healing data; and a communications system for transmitting the healing data to the data management system.

The control circuit described herein can comprise or be coupled to a control microcontroller unit. The control microcontroller unit is operably coupled to:
(1) the power management system to activate or deactivate power distribution to the device based on the prediction data outputted from the predictive algorithm;
(2) the electrodes to control application of the control voltages based on the prediction data outputted from the predictive algorithm; and
(3) the communication system to control transmission of healing data to the data management system.

### Example Data-Driven Computing (e.g., Machine Learning/Artificial Intelligence Methods)

The machine learning/artificial intelligence methods (e.g., adaptive machine learning methods) can be any method that enables closed loop control of the dose being delivered to the therapy site.

In one embodiment, the machine learning comprises a real-time adaptive ML-based feedback controller wherein the parameters of the machine learning algorithm should directly be adjusted to reduce the system's error, unlike the indirect control problem, where identification/estimation is used to approximate the system's model and then, the parameters of the ML-based controller are adjusted accordingly. One such example of an adaptive ML-based feedback controller comprises a class of artificial neural networks called the Radial Basis Function (RBF) network to achieve real-time online control without a priori knowledge of the dynamical model of the system and no dependency on large-scale datasets. The control scheme can be implemented on general-purpose computing systems. It uses the available information of the system (i.e., current inputs and the past states/outputs of the system, etc.) to adjust its parameters and decide the best controller output that maintains the system behavior to achieve the predefined goals (i.e., trajectory tracking, etc.) in an online manner. To do this, an adaptive external “sense and respond” learning algorithm is derived using adaptive Lyapunov-based methods, which are effective when dealing with unknown disturbances and unmodeled dynamics in an online fashion.

In one embodiment, to monitor and control the dynamics of healing state in real time, images of the therapy site are taken at regularly spaced time intervals. To set and maintain a specific healing state value, we control the system using a ML-based algorithm that maps changes in healing stare to prior control signals applied to the dose delivery system (e.g., pump). Using this information, the ML algorithm decides whether the healing state should be increased or decreased to achieve the desired healing state value and sends an updated control signal (voltage) to the delivery system, thus closing the control loop. The algorithm is not trained on any data a priori and makes no use of a model for either the bioelectronic device or the therapy/treatment site. Based on the target goal and current state, the parameters of the neural network are updated in between the time-lapse of the images such that the “learning” happens in real time and the target healing state is ultimately achieved. More specifically, the ML algorithm can leverage a neural network composed of an input layer, a hidden layer, and an output layer. The input layer receives the error value between the desired and the measured healing state values, information on prior to application of the control signals, as well as current and previous response of the therapy site to the applied control signals. The desired output consists of desired values at time k+1, k, and k−1, and the measured output consists of measured values at time k−1, k−2, and k−3 [2-3]. The hidden layer converges to a mapping that allows it to discern which value of the control signals should be applied to the individual proton pump surrounding the area of interest to achieve the desired healing state.

Any form of machine learning or artificial intelligence that can learn from changes in the healing state how to modify the control signals in a closed loop system can be used. Examples include, but are not limited to, neural networks (e.g., with one hidden layer or implementing radial basis functions), support vector machines, Bayesian networks, Hybrid systems, an MUA model that can be trained incrementally with new data as it becomes available, or able to implement unsupervised learning, reinforcement learning, or transfer learning.

In one embodiment, the healing state is quantified as a size of the therapy site comprising wound, i.e., quantifying the size of the wound and whether the wound is closing or opening (size of wound increasing or decreasing). In one or more embodiments, the rate of wound healing, i.e., change in wound size and rate of healing is predictive of ultimate healing status of the wound.

In another embodiment, the deep learning algorithm predicts the wound stage (for example, inflammation stage) and determines if additional directed therapy (e.g., anti-inflammatory drug) is needed to be delivered.

In one or more embodiments, the wound size/wound stage is determined using computer vision, e.g., using deep learning algorithms and traditional image processing methods [4]. One such machine learning method comprises detection of a wound in the image, cropping the wound area, segmentation of the wound area, post-processing (image processing), and measurement of the wound size. The machine learning can be trained on a training data sets annotated with correct identifications of the wound. Example machine learning algorithms include convolutional neural networks, support vector machines. Other forms of computer vision include, but are not limited to pattern recognition or object detection, although any computer implemented algorithm that can (e.g., automatically) identify and characterize a healing state (e.g., size, wound stage) of the wound or treatment site can be used.

In one or more embodiments, the algorithm interprets the wound stage (inflammation, proliferation, remodeling) and then tailors the therapy to move the wound to the next stage.

S However, the controlling described herein is not limited to machine learning and artificial intelligence. More generally, data-driven methods, or “real-time data-informed algorithms can be used.

### REFERENCES

The following references are incorporated by reference herein.
[1] Adv Wound Care (New Rochelle) 2020 September; 9(9):516-524, doi: 10.1089/wound.2019.1091. Epub 2020 Jan. 24. Development of a Model to Predict Healing of Chronic Wounds Within 12 Weeks, Sang Kyu Cho¹, Soeren Mattke², Hanna Gordon³, Mary Sheridan³, William Ennis^{3 4} PMID: 32941121, PMCID: PMC7522633, DOI: 10.1089/wound.2019.1091
[2] J. Selberg, M. Jafari, J. Mathews, M. Jia, P. Pansodtee, H. Dechiraju, C. Wu, S. Cordero, A. Flora, N. Yonas, S. Jannetty, M. Diberardinis, M. Teodorescu, M. Levin, M. Gomez, and M. Rolandi,
[3] Adv. Intell. Syst. 2, 2000140 (2020) and M. Jafari, G. Marquez, J. Selberg, M. Jia, H. Dechiraju, P. Pansodtee, M. Teodorescu, M. Rolandi, and M. Gomez, IEEE Control Syst. Lett. 5, 1133 (2020).
[4] H. Carrion, M. Jafari, M. D. Bagood, H. Y. Yang, R. R. Isseroff, M. Gomez, PLoS Comput Biol 2022, 18 (3), e1009852, https://doi.org/10.1371/journal.pcbi. 1009852.
[5] https://thesis.library.caltech.edu/10431/8/Kirchdoerfer_Trenton_2017_Thesis.pdf, data driven computing by Thomas Kirchdoerfer.
[6] Further information on one or more embodiments of the invention can be found in “A multi-ion electrophoretic pump for simultaneous on-chip delivery of H+, Na⁺, and Cl− Cite as: APL Mater. 10, 041112 (2022); doi: 10.1063/5.0084570 by Manping Jia, 1 Mohammad Jafari, 2,3 Pattawong Pansodtee, 1 Mircea Teodorescu, 1 Marcella Gomez, 2 and Marco Rolandi, and supplementary material.
[7] Further information on one or more embodiments of the present invention can be found in “Programmable Delivery of Fluoxetine via Wearable Bioelectronics for Wound Healing In Vivo Houpu Li, Hsin-ya Yang, Narges Asefifeyzabadi, Prabhat Baniya, Andrea Medina Lopez, Anthony Gallegos, Kan Zhu, Tiffany Nguyen, Cristian Hernandez, Ksenia Zlobina, Cynthia Recendez https://doi.org/10.1002/admt.202301115 Volume9, Issue 7 Apr. 4, 2024 2301115, and supplementary material.
[8] PCT publication No. WO 2023/196125 corresponding to PCT/US23/16245 filed Mar. 24, 2023, entitled “BIOELECTRONIC SMART BANDAGE FOR CONTROLLING WOUND PH THROUGH PROTON DELIVERY

### Hardware Environment

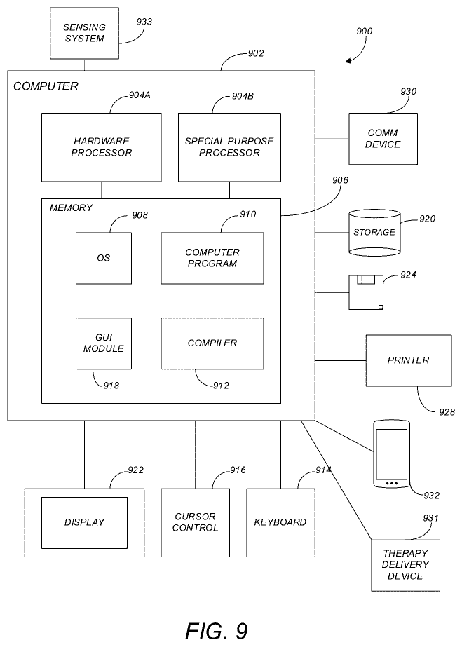

FIG. 9 is an exemplary hardware and software environment 900 (referred to as a computer-implemented system and/or computer-implemented method) used to implement one or more embodiments of the invention. The hardware and software environment includes a computer 902 and may include peripherals. Computer 902 may be a user/client computer, server computer, or may be a database computer. The computer 902 comprises a hardware processor 904A and/or a special purpose hardware processor 904B (hereinafter alternatively collectively referred to as processor 904) and a memory 906, such as random access memory (RAM). The computer 902 may be coupled to, and/or integrated with, other devices, including input/output (I/O) devices such as a keyboard 914, a cursor control device 916 (e.g., a mouse, a pointing device, pen and tablet, touch screen, multi-touch device, etc.) and a printer 928. In one or more embodiments, computer 902 may be coupled to, or may comprise, a portable or media viewing/listening device 932 (e.g., an MP3 player, IPOD, NOOK, portable digital video player, cellular device, personal digital assistant, etc.). In yet another embodiment, the computer 902 may comprise a multi-touch device, mobile phone, gaming system, internet enabled television, television set top box, or other internet enabled device executing on various platforms and operating systems.

In one embodiment, the computer 902 operates by the hardware processor 904A performing instructions defined by the computer program 910 (e.g., for implementing wound detection, determining healing state, closed loop control) under control of an operating system 908. The computer program 910 and/or the operating system 908 may be stored in the memory 906 and may interface with the user and/or other devices to accept input and commands and, based on such input and commands and the instructions defined by the computer program 910 and operating system 908, to provide output and results.

Output/results may be presented on the display 922 or provided to another device for presentation or further processing or action. The image may be provided through a graphical user interface (GUI) module 918. Although the GUI module 918 is depicted as a separate module, the instructions performing the GUI functions can be resident or distributed in the operating system 908, the computer program 910, or implemented with special purpose memory and processors.

In one or more embodiments, the display 922 is integrated with/into the computer 902 and comprises a multi-touch device having a touch sensing surface (e.g., track pod or touch screen) with the ability to recognize the presence of two or more points of contact with the surface. Examples of multi-touch devices include mobile devices (e.g., IPHONE, NEXUS S, DROID devices, etc.), tablet computers (e.g., IPAD, HP TOUCHPAD, SURFACE Devices, etc.), portable/handheld game/music/video player/console devices (e.g., IPOD TOUCH, MP3 players, NINTENDO SWITCH, PLAYSTATION PORTABLE, etc.), touch tables, and walls (e.g., where an image is projected through acrylic and/or glass, and the image is then backlit with LEDs).

Some or all of the operations performed by the computer 902 according to the computer program 910 instructions may be implemented in a special purpose processor 904B. In this embodiment, some or all of the computer program 910 instructions may be implemented via firmware instructions stared in a read only memory (ROM), a programmable read only memory (PROM) or flash memory within the special purpose processor 904B or in memory 906. The special purpose processor 904B may also be hardwired through circuit design to perform some or all of the operations to implement the present invention. Further, the special purpose processor 904B may be a hybrid processor, which includes dedicated circuitry for performing a subset of functions, and other circuits for performing more general functions such as responding to computer program 910 instructions. In one embodiment, the special purpose processor 904B is an application specific integrated circuit (ASIC), a field programmable gate array (FPGA), a graphics processing unit (GPU), or a processor adapted or configured to execute machine learning or artificial intelligence, or a processor adapted or configured for performing data-driven, or real-time data-informed algorithms.

The computer 902 may also implement a compiler 912 that allows an application or computer program 910 written in a programming language such as C, C++, Assembly, SQL, PYTHON, PROLOG, MATLAB, RUBY, RAILS, HASKELL, or other language to be translated into processor 904 readable code. Alternatively, the compiler 912 may be an interpreter that executes instructions/source code directly, translates source code into an intermediate representation that is executed, or that executes stored precompiled code. Such source code may be written in a variety of programming languages such as JAVA, JAVASCRIPT, PERL, BASIC, etc. After completion, the application or computer program 910 accesses and manipulates data accepted from I/O devices and stored in the memory 906 of the computer 902 using the relationships and logic that were generated using the compiler 912.

The computer 902 also optionally comprises an external communication device such as a modem, satellite link, Ethernet card, or other device for accepting input from, and providing output to, other computers 902.

In one embodiment, instructions implementing the operating system 908, the computer program 910, and the compiler 912 are tangibly embodied in a non-transitory computer-readable medium, e.g., data storage device 920, which could include one or more fixed or removable data storage devices, such as a zip drive, floppy disc drive 924, hard drive, CD-ROM drive, tape drive, etc. Further, the operating system 908 and the computer program 910 are comprised of computer program 910 instructions which, when accessed read and executed by the computer 902, cause the computer 902 to perform the steps necessary to implement and/or use the present invention or to load the program of instructions into a memory 906, thus creating a special purpose data structure causing the computer 902 to operate as a specially programmed computer executing the method steps described herein. Computer program 910 and/or operating instructions may also be tangibly embodied in memory 906 and/or data communications devices 930, thereby making a computer program product or article of manufacture according to the invention. As such, the terms “article of manufacture,” “program storage device,” and “computer program product,” as used herein, are intended to encompass a computer program accessible from any computer readable device or media.

Of course, those skilled in the art will recognize that any combination of the above components, or any number of different components, peripherals, and other devices, may be used with the computer 902.

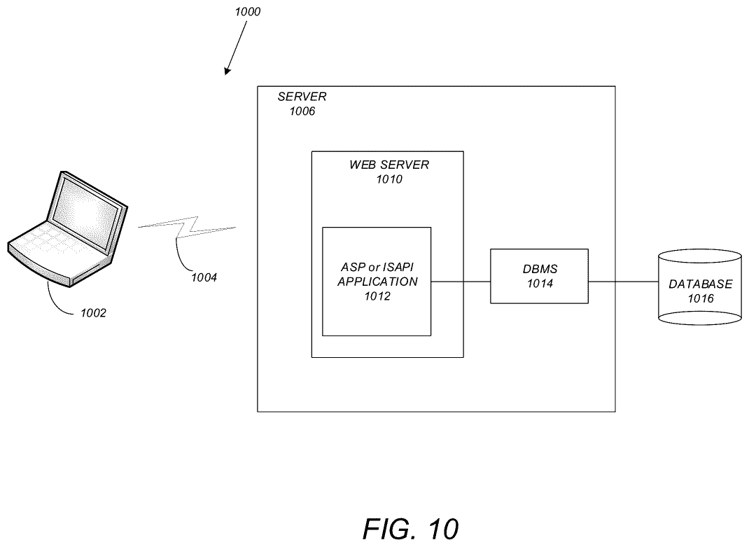

FIG. 10 schematically illustrates a typical distributed/cloud-based computer system 1000 using a network 1004 to connect client computers 1002 to server computers 1006. A typical combination of resources may include a network 1004 comprising the Internet, LANs (local area networks), WANs (wide area networks), SNA (systems network architecture) networks, or the like, clients 1002 that are personal computers or workstations (as set forth in FIG. 9), and severs 1006 that are personal computers, workstations, minicomputers, or mainframes (as set forth in FIG. 9). However, it may be noted that different networks such as a cellular network (e.g., GSM [global system for mobile communications] or otherwise), a satellite based network, or any other type of network may be used to connect clients 1002 and servers 1006 in accordance with embodiments of the invention.

A network 1004 such as the Internet connects clients 1002 to server computers 1006. Network 1004 may utilize ethernet, coaxial cable, wireless communications, radio frequency (RF), etc. to connect and provide the communication between clients 1002 and servers 1006. Further, in a cloud-based computing system, resources (e.g., storage, processors, applications, memory, infrastructure, etc.) in clients 1002 and server computers 1006 may be shared by clients 1002, server computers 1006, and users across one or more networks. Resources may be shared by multiple users and can be dynamically reallocated per demand. In this regard, cloud computing may be referred to as a model for enabling access to a shared pool of configurable computing resources.

Clients 1002 may execute a client application or web browser and communicate with server computers 1006 executing web servers 1010. Such a web browser is typically a program such as MICROSOFT INTERNET EXPLORER/EDGE, MOZILLA FIREFOX, OPERA, APPLE SAFARI, GOOGLE CHROME, etc. Further, the software executing on clients 1002 may be downloaded from server computer 1006 to client computers 1002 and installed as a plug-in or ACTIVEX control of a web browser. Accordingly, clients 1002 may utilize ACTIVEX components/component object model (COM) or distributed COM (DCOM) components to provide a user interface on a display of client 1002. The web server 1010 is typically a program such as MICROSOFT'S INTERNET INFORMATION SERVER.

Web server 1010 may host an Active Server Page (ASP) or Internet Server Application Programming Interface (ISAPI) application 1012, which may be executing scripts. The scripts invoke objects that execute business logic (referred to as business objects). The business objects then manipulate data in database 1016 through a database management system (DBMS) 1014. Alternatively, database 1016 may be part of, or connected directly to, client 1002 instead of communicating/obtaining the information from database 1016 across network 1004. When a developer encapsulates the business functionality into objects, the system may be referred to as a component object model (COM) system. Accordingly, the scripts executing on web server 1010 (and/or application 1012) invoke COM objects that implement the business logic. Further, server 1006 may utilize MICROSOFT'S TRANSACTION SERVER (MTS) to access required data stored in database 1016 via an interface such as ADO (Active Data Objects), OLE DB (Object Linking and Embedding DataBase), or ODBC (Open DataBase Connectivity).

Generally, these components 1000-1016 all comprise logic and/or data that is embodied in/or retrievable from device, medium, signal, or carrier, e.g., a data storage device, a data communications device, a remote computer or device coupled to the computer via a network or via another data communications device, etc. Moreover, this logic and/or data, when read, executed, and/or interpreted, results in the steps necessary to implement and/or use the present invention being performed.

Although the terms “user computer”, “client computer”, and/or “server computer” are referred to herein, it is understood that such computers 1002 and 1006 may be interchangeable and may further include thin client devices with limited or full processing capabilities, portable devices such as cell phones, notebook computers, pocket computers, multi-touch devices, and/or any other devices with suitable processing, communication, and input/output capability.

Of course, those skilled in the art will recognize that any combination of the above components, or any number of different components, peripherals, and other devices, may be used with computers 1002 and 1006. Embodiments of the invention are implemented as a software/application an a client 1002 or server computer 1006. Further, as described above, the client 1002 or server computer 1006 may comprise a thin client device or a portable device that has a multi-touch-based display.

### Process Steps

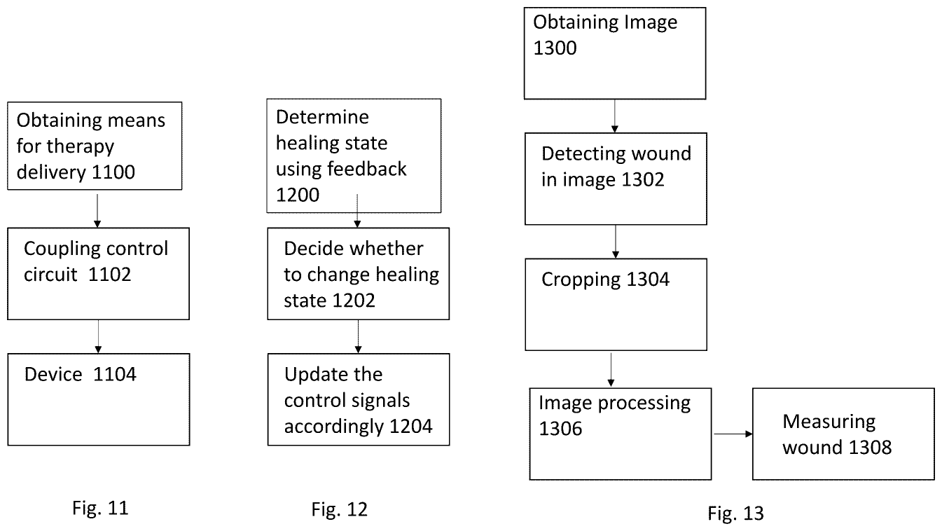

FIG. 11 illustrates a method of manufacturing a system or device for delivering therapy to a therapy site.
Block 1100 represents providing means for delivering a dose of a therapy to a treatment site;

Block 1102 represents providing one or more sensors (e.g., sensor system, an imaging system, or camera, or microscope) configured for sensing data describing/representing/associated with/corresponding to one or more properties (e.g., a healing state) of the treatment site and outputting sensing data in response thereto; and

Block 1104 represents coupling a computer configured to execute a machine learning (e.g., algorithm) determining a healing state of the treatment site from the data and determining the dose of the therapy in response to the healing state.

Block 1106 represents the end result, a device or system. The device or system can be implemented in many ways, including but not limited to, the following (referring also to the FIGS. 1-13).
1. A system, device, or apparatus 100, 400, 900 for delivering therapy to a treatment site, comprising:
means 931 for delivering a dose 104, 404 of a therapy 105, 406 to a treatment/therapy site 106, 408;
a sensor 933 (e.g., an imaging system 108) configured for sensing the treatment site (e.g., capturing one or more images 200, 600 of the treatment site) and outputting sensor data (e.g., image data) in response thereto (the image data or sensor data representing, associated with, describing, or corresponding to a property of the treatment site from which a healing state can be derived); and
wherein the means comprises or is coupled or connected to a computer 902 configured to execute a machine learning (e.g., a machine learning algorithm) determining a healing state of the treatment site from the image data and determining the dose of the therapy in response to the healing state.
2. A system for delivering therapy to a treatment site, comprising:
means 931 for delivering a dose 104, 404 of a therapy 105, 406 to a treatment site 106, 408;
a sensor 933 for sensing (e.g., imaging system or camera configured for capturing one or more images of) the treatment site and outputting (e.g., image) data in response thereto (e.g., the image data or sensor data representing, associated with, describing, or corresponding to a property of the treatment site from which a healing state/status can be derived); and
a data-driven controller 902 configured to (e.g., execute machine learning, e.g., a machine learning algorithm) to control the dose in a closed loop, e.g., using data driven computing and/or a (e.g., real time) data driven or data-informed algorithm, by:
determining a healing state of the treatment site from the data, and
using the healing state as feedback to update the dose delivered to the treatment site so that the therapy increases healing, and/or a rate of the healing, of the treatment site and/or the healing state converges to a desired healing state.
3. The system of clause 1 or 2, wherein the means for delivering the dose comprises a control circuit 410 coupled to a microfluidic device or pump 402, 102 operable to pump the therapy 105 to the treatment site in response to control signals (e.g., voltages V1-V4, V_{Flx}) received from the control circuit.
4. The system of clause 3, wherein the control circuit comprises or is coupled to the computer executing the machine learning, or wherein the controller is coupled to or comprises the control circuit, so that the machine learning and the control circuit implement a closed loop control of the control signals, wherein the machine learning:
maps changes in image data to the control signals previously applied,
makes a decision whether the healing state should be changed to achieve a desired healing state,
and updates the control signal applied to the microfluidic device/pump via the control circuit if necessary in response to the decision.
5. The system of any of the clauses 1-4, wherein the machine learning is executed in a neural network 300 and the parameters of the neural network, comprising the bias and weights applied at each of the one or more layers of the neural network, are updated during a time-lapse between the image frames from which the images data is generated, such that the machine learning algorithm learns how to adapt the control signals in real time to obtain the desired healing state.
6. The system of any of the clauses 1-5, wherein the healing state comprises a size 602 of the treatment site comprising a wound 409, wherein the computer executes a computer vision algorithm to identify and calculate the size of the wound so that the therapy reduces a size (e.g., diameter) of the wound 409.
7. The system of any of the clauses 1-6, wherein the computer/controller and/or the control circuit are coupled to provide closed-loop control of the dose delivered to the treatment site in response to feedback comprising the healing state.
8. The system of any of the clauses 1-7, wherein the means for delivery is integrated with a wound dermal interface 412, 118 configured for attachment to the treatment site comprising the wound 409 and the computer or controller is integrated with the wound dermal interface or wirelessly connected to the means for delivery.
9. The system of any of the clauses 1-8, wherein the means for delivering the dose comprises:
   a control circuit 410, 114 programmable to output voltage control signals V1-V4 controlling the dose of ions according to a delivery profile 500 for treating a treatment site; and
   an ion pumping system 102 comprising a plurality of ion channels 110 and a plurality of electrodes 112, 458 coupled to the control circuit, wherein the ions are pumped through the ion channels to the treatment site in response to the voltage control signals.
10. the device of clause 9, wherein the ion pumping system further comprises: a housing 116, 502 for a plurality of the channels 110, each of the channels comprising:
   a reservoir 120 storing a fluid 122 comprising the ions;
   a reference electrode 124 electrically connected to the fluid,
an array of control electrodes 112 each comprising an end 126 for positioning at different spatial locations 128 at the treatment site;
one of the ion channels 110 connecting the reservoir to the ends of the control electrodes, the one of the ion channels containing an ion conducting material 130 for conducting the ions; and
wherein the control circuit is operable to activate the pumping of the ions 132 to one or more predetermined locations in the treatment site, by applying one or more of the voltage control signals between the reference electrode and one or more of the control electrodes associated with the predetermined locations according to the delivery profile.
11. The device of clause 9 or 10, wherein the ions in each of the channels and reservoirs comprise different ionic species and/or different polarities.
12. A system comprising the device of any of the clauses 1-11, comprising a computer or the controller coupled to and/or comprising the control circuit, wherein the computer or controller 902 comprises one or more processors configured to execute machine learning (e.g., a machine learning algorithm) determining the voltage control signals for delivery of the ions according to the delivery profile comprising the predetermined locations according and/or a predetermined temporal profile.
13. The system of clause 12, further comprising the imaging system 933 coupled to the computer or the controller and configured to capture the image 200, 600 of a spatial distribution of the dose, or the ion flux, as a function of time, and output image data in response thereto, wherein the machine learning algorithm updates the voltage control signals V1, V2, V3, VFlx using the image data as feedback and in a closed loop.
14. The device or system of any of the clauses 10-13, wherein the housing 116 comprises:
a substrate 140;
metallization traces 142 comprising the control electrodes on the substrate; a first insulation layer 144 over the metallization traces with first openings exposing the ends of the traces for contact with the treatment site;
a polymer layer on the first insulation layer patterned with second openings defining the ion channels;
a second insulation layer on the polymer layer forming a cover over the ion channels and comprising second openings exposing the ends of the traces in the first openings;
a layer on the second insulation layer comprising cavities forming the reservoirs 120 and a target opening exposing the treatment site.
15. the device or system of any of the clauses 3-14, further comprising a battery 504 coupled to the control circuit for powering the device, wherein the control circuit 410 further comprises:
   a microcontroller or processor 450 (e.g., comprising or coupled to an analog to digital converter, ADC);
   a digital to analog converter (DAC);
   a memory 452, and
   a program stored in the memory and executed by the microcontroller or processor for commanding the DAC to output the voltage control signals to the electrodes so as to drive a current of the ions through the ion conducting material to and/or from the treatment site.
16. the device of clause 15, wherein:
the control circuit further comprises one or more resistors 456 connected for sensing a current associated with pumping of the ions and used to measure the dose, so that:
the current flowing through the resistors generates sense voltages used to measure the dose, and
the sense voltages can be read by an analog to digital controller in the microcontroller/processor 450 or by external probes.
17. The device or system of any of the clauses 9-16, wherein the wearable ion pumping system further comprises:
a housing 502 housing:
   reservoirs 504 storing the fluid 506 comprising the ions 508, and the ion channels 510, the ion channels each loaded with an ion conducting material 512 between the reservoir and the treatment site; and
the electrodes 458 electrically connected to the fluid and the control circuit so that the electrodes activate the pumping by applying the voltage control signals VFIx to the fluid.
18. The device or system of any of the clauses 9-17, wherein:
the plurality n of the electrodes 458 each comprise a pin 460 comprising a first end electrically coupled to a wire 462,
the housing consists essentially of a polymer and comprises:
   n cavities 464 forming the plurality n of the reservoirs;
   n through holes 466 through which the pins are inserted into the reservoirs, positioned so that, for 1≤i≤n, the i^{th} one of the reservoirs is paired with the i^{th} one of the electrodes and the i^{th} one of the first ends may electrical connect to the i^{th} one of the wires disposed in the i^{th} one of the reservoirs; and
   n mounts 468 comprising openings through which the ion channels are inserted and mounting an i^{th} first channel end of the i^{th} one of the ion channels in fluidic connection with the fluid the i^{th} one of the reservoirs, so that an i^{th} second channel end of the i^{th} one of the channels can be in physical contact with the treatment site; and
the device further comprises a printed circuit board 470 physically attached to the housing and comprising the control circuit 410 soldered to the second ends 470 of the pins 460.
19. The device or system of any of the clauses 9-18, wherein:
the voltage control signals apply a bias across first one of the electrodes 458a in a first one of the reservoirs and a second one of the electrodes 458b in a second one of the reservoirs, to drive a:
flow 550 of a first type of the ions, having a first polarity type, from the first one of the reservoirs to the treatment site through a first one of the ion channels, and
a return flow 550b of a second type of the ions from the treatment site and having the first polarity type, to the second one of the reservoirs via a second one of the ion channels, and
the ion channels comprise an ion exchange membrane allowing the flow of the ions of the first polarity type to and from the treatment site but blocking flow of ions or charge having a second polarity type (opposite the first polarity type);
the first one of the electrodes comprises a working electrode/anode and the second one of the electrodes comprises a counter electrode/cathode, and
the voltage control signals drive an electrochemical reaction at the electrodes, and the electrochemical reaction:
oxidizes the working electrode to release an electron and the first type of the ions comprising the first polarity type; and
consumes an electron at the counter electrode to release a charge having a second polarity type (opposite the first polarity type) that pairs or charge balances with the second type of ions comprising physiological ions.
20. The device or system of any of the clauses 9-19, wherein the fluid comprises a solution comprising a biochemical or drug ionized (e.g., by protonation) by the solution, so as to form the first type of ions comprising biomolecules or drugs.
21. The device or system of clause 20, wherein the biochemical comprises fluoxetine.
22. An intelligent wound care management system 800 comprising the system of any of the clauses 1-21, comprising:
a wound dermal interface 802 for attaching the device to the treatment site;
the wound sensor (e.g., imaging system 804) coupled to the treatment site positioned for measuring the dose and/or a healing state of the treatment site and outputting healing data in response thereto;
an alarm system 806 coupled to the sensors, the alarm system comprising one or more processors configured for determining whether the healing data is within an acceptable range for the treatment and outputting an alarm signal indicating whether the healing data is within the acceptable range or not;
the computer 808, 902 comprising one or more processors configured for executing the machine learning algorithm to determine scheduling of the control voltages applied to the electrodes in response to the alarm signal and outputting prediction data;
a power management system 810 comprising a power source coupled to the device, for distributing power to the device;
a data management system 812 configured for storing the healing data;
a communications system 814 for transmitting the healing data to the data management system; and
the control circuit comprising or coupled to a control microcontroller unit, the control microcontroller unit operably coupled to:
   the power management system to activate or deactivate power distribution to the device based on the prediction data outputted from the data driven (e.g., predictive) algorithm;
   the electrodes to control application of the control voltages based on the prediction data outputted from the data-driven (e.g., predictive) algorithm;
   the communication system to control transmission of healing data to the data management system.
23. The system of any of the clauses 1-22 operable to control delivery of the dose so that the ions cause re-epithelialization of the treatment site comprising a wound, as characterized by the dose causing transitioning of macrophages in the treatment site to an anti-inflammatory pro-reparative phonotype (away from an inflammatory phenotype) early in the treatment cycle.
24. The system of any of the clauses 1-23, wherein the wound dermal interface comprises a bandage, dressing, adhesive, patch, or other mechanism for attaching the device to the treatment site and/or covering the treatment site.
25. The system of any of the clauses 22-24, wherein the power management system includes a battery and is configured to power the device for at least a week with continuous delivery of the ions.
26. The system of any of the clauses 1-25, wherein examples of the therapy include, but are not limited to ions (e.g., H+, Na⁺, K+, or Cl−), biomolecules, drugs, chemical species, charged species, protons, or electric fields or potentials.
27. The system of any of the clauses 1-26, comprising a (e.g., wearable) bioelectronic device or bioelectronic bandage comprising the means and the controller.
28. The system of any of the clauses 1-27, comprising an integrated circuit comprising the means and the controller.
29. The system of any of the clauses 1-28, wherein the controller comprises a computer (e.g., comprising one or more processors) or one or more circuits.
30. The system of clause 29, wherein the computer comprises one or more processors; one or more memories; and an application/program stored in the one or more memories, wherein the application executed by the one or more processors performs the machine learning/artificial intelligence and/or control functions described herein.
31. The system of clause 29 or 30, wherein the computer comprises an integrated circuit (e.g., an application specific integrated circuit) and the machine learning is implemented by the hardware of the circuit.
32. The system of any of the clauses 1-31, wherein the treatment site comprises a wound including, but not limited to, an acute surgical wound, a blast trauma wound, a chronic wound such as, but not limited to, a diabetic ulcer, venous ulcer, or pressure ulcer.
33. The system of any of the clauses 1-32, wherein the treatment site comprises an injury or wound an a human or animal, the wound having any size and/or geometry and the device being sized or adapted to attach to and deliver the therapy to the wound of any size or geometry.
34. The system of any of the clauses 1-33, wherein the machine learning learns how to
update the dose and the control signals in real time and/or during a time lapse
between the image frames from which the image data is obtained.
35. The system of any of the clauses 1-34, wherein:
the means delivers the dose in response to control signals (e.g., current);
the computer comprises a hardware control circuit (e.g., ASIC) executing the machine learning (e.g., reinforcementlearning), and
the machine learning:
   determines a reference signal 302 representing the desired healing state; and
   updates the control signals using the hardware without an algorithm by comparing the image data to the reference signal.
36. The system of any of the clauses 1-35, wherein the controller or the control circuit automatically control the sensor to capture the data and the means to deliver the therapy in automatic response to the feedback.
37. The system of any of the clauses 1-36, wherein the dose is controlled by the dosed loop in real time in response to the sensor data (e.g., as the sensor data is received), and the machine learning learns how to control the dose in real time.
38. The system of any of the clauses 1-37, wherein the data-driven controller or data-driven computer executes or performs data-driven computing or an data-informed algorithm or data-driven algorithm including, but not limited to, a computing or algorithm approach where decisions, processes, and/or outcomes are based on analysis of the data, wherein analyzing the data allows prediction and/or optimization of the dose. Examples include, but are not limited to, machine learning and/or artificial intelligence or a predictive algorithm.
39. The system of my of the clauses 1-38, wherein the system comprises or is a (e.g., wearable) device comprising at least one of the means, the sensor, or the controller, e.g., wearable by a human.
40. The system of my of the clauses 1-38, wherein the system comprises or is a (e.g., wearable) (e.g., electronic device, optoelectronic device, bioelectronic device or integrated device or circuit) device comprising the means, the senior, or the controller, e.g., wearable by a human or animal.
41. A method, comprising:
   using data driven computing or a data-driven algorithm to output control signals used to control a dose of therapy delivered to a treatment site in a closed loop by:
   determining a healing state of the treatment site from data obtained from a sensor sensing the treatment site, and
   using the healing state as feedback to determine the control signals used to control the dose delivered to the treatment site so that the therapy increases healing of the treatment site and/or the healing state converges to a desired healing state.
42. The method of clause 41, wherein the data-driven computing comprises machine learning executed in software or hardware, and further comprising delivering the dose by pumping the therapy comprising ions to the treatment site.
43. The method of clause 41 or 42, wherein the data-driven computing executes a real-time data informed algorithm for determining the healing state and the dose in real time as the data is updated and received from the sensor.
44. The method my of the clauses 41-43 performed using the device or system of any of the clauses 1-41.
45. The method or device of my of the clauses 1-44, wherein the means for delivery comprises a pump, a microfluidic device, n ion pump, or equivalents thereof.
46. The method or device of ay of the clauses 1-45, wherein treatment site comprises living (e.g., biological) tissue or biological cells or in-vivo tissue, e.g., on a human or animal, e.g., on a limb or body of the human or animal.

FIG. 12 is a flowchart illustrating a method of performing data driven computing comprising determining 1200 healing state using feedback from sensor, deciding 1202 whether to change healing state, and updating 1204 the control signals accordingly.

FIG. 13 is a flowchart illustrating a method of wound detection comprising obtaining image 1300, detecting wound in image 1302, cropping 1304, and image processing 1306, and measuring wound 1308.

Methods of FIG. 12 and FIG. 13 can be implemented using any of the device of clauses 1-46.

### CONCLUSION

This concludes the description of the preferred embodiment of the present invention. The foregoing description of one or more embodiments of the invention has been presented for the purposes of illustration and description. It is not intended to be exhaustive or to limit the invention to the precise form disclosed. Many modifications and variations are possible in light of the above teaching. It is intended that the scope of the invention be limited not by this detailed description, but rather by the claims appended hereto.

What is claimed is:

1. A system for delivering therapy to a treatment site, comprising:
means for delivering a dose of a therapy to a treatment site;
a sensor configured for sensing the treatment site and outputting data in response thereto; and
a data-driven controller or data-driven computer configured to control the dose in a closed loop by:
determining a healing state of the treatment site from the data, and
using the healing state as feedback to update or determine the dose delivered to the treatment site so that the therapy increases a rate of healing of the treatment site and/or the healing state converges to a desired healing state.

2. The system of claim 1, wherein the controller implements a real-time data informed algorithm for determining the healing state and the dose in real time as the data is updated and received from the sensor.

3. The system of claim 1, wherein the data-driven controller or data-driven computer is configured to execute machine learning or artificial intelligence to determine the healing state and the dose using the data as the feedback.

4. The system of claim 1, wherein:
the means for delivering the dose comprises a control circuit coupled to a pump operable to pump the therapy to the treatment site in response to one or more control signals received from the control circuit, and
the controller comprises or is coupled to the control circuit.

5. The system of claim 4, wherein the machine learning:
maps changes in image data to the control signals previously applied,
makes a decision whether the healing state should be changed to achieve the desired healing state,
and updates the control signals applied to the pump via the control circuit if necessary in response to the decision, and
the machine learning is executed in a neural network and the parameters of the neural network, comprising the bias and weights applied at each of the one or more layers of the neural network, are updated during a time-lapse between the image frames from which the images data is generated, such that the machine learning learns how to adapt the control signals in real time to obtain the desired healing state.

6. The system of claim 4, wherein:
the treatment site comprises a wound,
the healing state comprises at least one of a change in wound size or a rate of healing of the wound, and
the controller executes a computer vision algorithm to identify and calculate at least one of the size and the rate of healing of the wound.

7. The system of claim 4, wherein the machine learning learns how to update the
dose and the control signals in real time and/or during a time lapse between the
image frames from which the image data is obtained.

8. The system of claim 1, wherein:
the means delivers the dose in response to control signals;
the computer or controller comprises a hardware control circuit executing the machine learning, and
the machine learning:
determines a reference signal representing the desired healing state; and
updates the control signals using the hardware without an algorithm by comparing the image data to the reference signal.

9. The system of claim 1, wherein the means for delivering the dose comprises:
a control circuit programmable to output voltage control signals controlling the dose
of ions according to delivery profile for treating a treatment site; and
an ion pumping system comprising a plurality of ion channels and a plurality of electrodes coupled to the control circuit, wherein the ions are pumped through the ion channels to the treatment site in response to the voltage control signals.

10. The device of claim 9, wherein the ion pumping system further comprises:
a housing for a plurality of the channels, each of the channels comprising:
a reservoir storing a fluid comprising the ions;
a reference electrode electrically connected to the fluid;
an array of control electrodes each comprising an end for positioning at different spatial locations at the treatment site;
one of the ion channels connecting the reservoir to the ends of the control electrodes, the one of the ion channels containing an ion conducting material for conducting the ions; and
wherein the control circuit is operable to activate the pumping of the ions to one or more predetermined locations in the treatment site, by applying one or more of the voltage control signals between the reference electrode and one or more of the control electrodes associated with the predetermined locations according to the delivery profile.

11. The device of claim 9, further comprising a battery coupled to the control circuit for powering the device, wherein the control circuit further comprises:
a microcontroller or processor;
a digital to analog converter (DAC);
a memory, and
a program stored in the memory and executed by the microcontroller or processor for commanding the DAC to output the voltage control signals to the electrodes so as to drive a current of the ions through the ion conducting material to and/or from the treatment site.

12. The device of claim 11, wherein:
the control circuit further comprises one or more resistors connected for sensing a current associated with pumping of the ions and used to measure the dose, so that:
the current flowing through the resistors generates sense voltages used to measure the dose, and
the sense voltages can be read by an analog to digital controller in the microcontroller/processor or by external probes.

13. The device of claim 9, wherein the wearable ion pumping system further comprises:
a housing housing:
reservoirs storing the fluid comprising the ions, and
the ion channels, the ion channels each loaded with an ion conducting material
between the reservoir and the treatment site; and
the electrodes electrically connected to the fluid and the control circuit so that the electrodes activate the pumping by applying the voltage control signals to the fluid; and
a printed circuit board physically attached to the housing and comprising the control circuit connected to the electrodes.

14. The device of claim 13, wherein:
the voltage control signals apply a bias across first one of the electrodes in a first one of the reservoirs and a second one of the electrodes in a second one of the reservoirs, to drive a:
flow of a first type of the ions, having a first polarity type, from the first one of the reservoirs to the treatment site through a first one of the ion channels, and
a return flow of a second type of the ions from the treatment site and having the first polarity type, to the second one of the reservoirs via a second one of the ion channels, and
the ion channels comprise an ion exchange membrane allowing the flow of the ions of the first polarity type to and from the treatment site but blocking flow of ions or charge having a second polarity type (opposite the first polarity type);
the first one of the electrodes comprises a working electrode/anode and the second one of the electrodes comprises a counter electrode/cathode, and
the voltage control signals drive an electrochemical reaction at the electrodes, and the electrochemical reaction:
oxidizes the working electrode to release an electron and the first type of the ions comprising the first polarity type; and
consumes an electron at the counter electrode to release a charge having a second polarity type (opposite the first polarity type) that pairs or charge balances with the second type of ions comprising physiological ions.

15. The device of claim 13, wherein the fluid comprises a solution comprising a biochemical or drug ionized (e.g., by protonation) by the solution, so as to form the first type of ions comprising biomolecules or drugs.

16. An intelligent wound care management system comprising the system of claim 1, comprising:
a wound dermal interface for attaching the device to the treatment site;
the imaging system coupled to the treatment site positioned for measuring the dose and/or a healing state of the treatment site and outputting healing data in response thereto;
an alarm system coupled to the sensors, the alarm system comprising one or more processors configured for determining whether the healing data is within an acceptable range for the treatment and outputting an alarm signal indicating whether the healing data is within the acceptable range or not;
the computer comprising one or more processors configured for executing the machine learning to determine scheduling of the control voltages applied to the electrodes in response to the alarm signal and outputting prediction data;
a power management system comprising a power source coupled to the device, for distributing power to the device;
a data management system configured for storing the healing data;
a communications system for transmitting the healing data to the data management system; and
the control circuit comprising or coupled to a control microcontroller unit, the control microcontroller unit operably coupled to:
the power management system to activate or deactivate power distribution to the device based on the prediction data outputted from the predictive algorithm;
the electrodes to control application of the control voltages based on the prediction data outputted from the predictive algorithm;
the communication system to control transmission of healing data to the data management system.

17. The system of claim 1 operable to control delivery of the dose so that the ions cause re-epithelialization of the treatment site comprising a wound, as characterized by the dose causing transitioning of macrophages in the treatment site to an anti-inflammatory pro-reparative phonotype (away from an inflammatory phenotype) early in the treatment cycle.

18. The system of claim 1, wherein the wound dermal interface comprises a bandage, dressing, adhesive, patch, or other mechanism for attaching the device to the treatment site and/or covering the treatment site.

19. A method, comprising:
using data driven computing or a data-driven algorithm to output control signals used to control a dose of therapy delivered to a treatment site in a closed loop by:
determining a healing state of the treatment site from data obtained from a sensor sensing the treatment site, and
using the healing state as feedback to determine the control signals used to control the dose delivered to the treatment site so that the therapy increases healing of the treatment site and/or the healing state converges to a desired healing state.

20. The method of claim 19, wherein the data-driven computing comprises machine learning executed in software or hardware, and further comprising delivering the dose by pumping the therapy comprising ions to the treatment site.
